(19) United States
(12) Patent Application Publication
(10) Pub. No.: US 2025/0302467 A1
(43) Pub. Date: Oct. 2, 2025

(54) SUTURING DEVICES, AND THERAPEUTIC APPARATUSES AND THERAPEUTIC DEVICES WITH SUTURING DEVICES

(71) Applicants: Ping WAN, Shanghai (CN); SCARLESS MEDICAL TECHNOLOGY (JIANGSU) CO., LTD, Changzhou (CN)

(72) Inventor: Ping WAN, Shanghai (CN)

(73) Assignees: Ping WAN, Shanghai (CN); SCARLESS MEDICAL TECHNOLOGY (JIANGSU) CO., LTD, Changzhou (CN)

(21) Appl. No.: 19/238,515

(22) Filed: Jun. 15, 2025

Related U.S. Application Data
(63) Continuation of application No. PCT/CN2023/070089, filed on Jan. 3, 2023.

(51) Int. Cl. A61B 17/04; A61B 1/00; A61B 17/00; A61B 17/32; A61B 34/00

(52) U.S. Cl. CPC A61B 17/0469; A61B 1/00087; A61B 1/00133; A61B 17/0491; A61B 17/320016; A61B 34/71; A61B 2017/00349

(57) ABSTRACT

The present disclosure relates to a suturing device, and a therapeutic apparatus and a therapeutic device with the suturing device. The suturing device includes a main housing, a suture needle, a needle insertion assembly, and an anti-retraction component. The needle insertion assembly is disposed within the main housing, including a drive component, a return component, and a needle insertion tooth. The needle insertion tooth is in a separable engagement relationship with the suture needle. When the drive component acts on the needle insertion tooth and the suture needle that are in an engagement state, the suture needle is controlled to be inserted, and the return component undergoes deformation when the suture needle is inserted. When the drive component is released, the engagement state of the needle insertion tooth and the suture needle is released, and the return component drives the needle insertion tooth to return.

### CROSS-REFERENCE TO RELATED APPLICATIONS

This application is a continuation of International Patent Application No. PCT/CN2023/070089, filed on Jan. 3, 2023, the contents of which are hereby incorporated by reference.

### TECHNICAL FIELD

The present disclosure relates to a therapeutic apparatus, and in particular to a suturing device, and a therapeutic apparatus and a therapeutic device with the suturing device.

### BACKGROUND

Minimally invasive technology is a trend in the development of modern surgery, but it still leaves scars on the patient's body surface. As a result, scarless surgery emerged. Natural Orifice Transluminal Endoscopic Surgery (NOTES) becomes a key research focus as it must be performed using flexible endoscopes via natural orifices, and this approach marks the third-generation surgical evolution following open and laparoscopic techniques. Scarless surgery on the body surface has minimal physiological and psychological trauma, and correspondingly reduces the occurrence of intraoperative and postoperative complications. Additionally, NOTES does not require general anesthesia and requires less use of anesthetic drugs, greatly reducing the risk of anesthesia, the postoperative recovery is faster, and the recovery time is significantly shortened, reducing the occurrence of postoperative complications, which is more favorable for extremely weak patients and patients with higher surgical risks.

Although the endoscopic helix tacking system can suture larger wounds or other target suturing tissues, the system not only has many accessories and complex operations, but also relies on at least three metal helical nails for suturing, and it still relies on clips for knotting. This system, in which the helical nails that are rotated through the tissues for suturing remain in the body, makes it difficult to rule out the possibility of secondary damage to the digestive system, such as perforation, intestinal leakage, and adhesions, or even to the urinary and reproductive systems. At the same time, the helical nails may accumulate with the knotted rigid clips, making it easier for the peristalsis of the digestive tract and the movement of the abdomen to cause bodily injury.

Patent No. CN111419312A discloses suturing devices, therapeutic apparatuses with suturing devices, and therapeutic systems. The patent achieves suturing and knotting of soft tissues in the body by controlling the insertion of a needle insertion assembly. The needle insertion assembly of the suturing device in this patent has two manipulation cords (ropes), which are controlled to move the needle insertion assembly in different directions during operation, thereby achieving needle insertion, extraction, and threading. The installation of two maneuvering cords (ropes) on the control handle makes the structure of the suturing device and the control handle complex, the connection complicated, and the steps increased, resulting in reduced operator compliance, increased volume at the connection, and increased probability of thread detachment during operation. The control of the two manipulating ropes makes the control action of the control handle complicated and easily chaotic. The control of the two manipulating ropes, in turn, requires both hands of the operator, and the right hand of the operator of the endoscope is the main and continuous operating hand. If the right hand is called upon to cooperate with the left hand in the operation of the control handle, it may have adverse effects on the progress, speed, continuity, accuracy, safety, sustainability, and attention of using the endoscope and the suturing device for treatment or examination. Additionally, if the direction change of the manipulating rope within the control handle or the suturing device relies on components with right angles or other non-curved smooth interfaces, it may lead to increased resistance to the movement of the manipulating rope, difficulty in pulling back the pull rod with fingers, inaccurate needle insertion, easy tissue damage, fatigue fracture of the manipulating rope, wear of the endoscope lumen or forceps channel, and a decrease in the number of times the test pull rod is pulled back.

The setting of the two manipulation cords (ropes) completely blocks the possibility of turning the C-shaped opening of the suturing device. Once the suturing device rotates coaxially, the two cords (ropes) will be cross-twisted, the needle will not be able to move back and forth, and the operation will stop abruptly, thus, the suturing cannot be performed. Due to the suturing device in the patent is unable to rotate coaxially to arbitrarily convert the direction and position of the C-shaped opening, it can only rely on the bending of the apex of the endoscope to locally adjust the direction and position of suturing, especially in narrow internal spaces, which increases the difficulty of suturing, weakens the strength of suturing, and cannot quickly and accurately align well or even suture the target tissue.

When the endoscopic suturing device enters the body for suturing, the operator's field of view completely depends on the lens at the apex of the endoscope. Due to the apex of the endoscope being attached to the suturing device, the operations of the needle insertion, suturing, and knotting rely on the manipulating rope, which is constantly moving back and forth and does not have a high degree of elasticity. The experiments have shown that this inevitably leads to the accumulation of manipulation ropes in front of the lens or an increase in the movement of the thread. To avoid this situation, a torque limiter is installed inside the control handle to prevent the manipulating rope from breaking. However, this not only increases the production and manufacturing costs, but also results in an increase in the volume of the control handle. The distance between the pull rod of the control handle and the base of the thumb of the operator is too large, which is not in line with ergonomics and makes the operator's fingers easily fatigued, affecting the progress of the operation. Twisting, pulling out the circular knob of the control handle, and other actions of operating the control handle require the assistance of another hand.

Due to the varying diameters of endoscopes, if the diameter of the tubular portion at the apex of the endoscope that is connected to the suturing device is fixed, it is difficult to apply to the endoscopes of a plurality of diameters, or it limits the applicability of one specification of the suturing device, or increases the production cost of multiple specifications of the suturing device, or passively adapt to endoscopes of new diameters after installation. The connecting component between the tubular portion and the main housing is a curved tile-like plate, which can limit the adjustment of the diameter of the tubular portion.

Therefore, it is desired to propose a suturing device, a therapeutic apparatus and a therapeutic device with the suturing device, so that the insertion of the suture needle can be realized solely by controlling the drive component through a single cord (rope) control handle, and during the procedure, the operator of the endoscope only needs to control the control handle with one hand, while the other hand can continue to suture. The operation of a single component also allows the operator to control the control handle without sight, ensuring that the operator's attention is always focused on the operation of the endoscope, thereby ensuring the safety and continuity of the surgical operations.

### SUMMARY

One or more embodiments of the present disclosure provide a suturing device, comprising: a main housing; a suture needle slidingly disposed within the main housing, wherein one end of the suture needle connects to a suture thread and the other end of the suture needle is a tip; a needle insertion assembly disposed within the main housing, the needle insertion assembly including a drive component, a return component, and a needle insertion tooth, the needle insertion tooth being in a separable engagement relationship with the suture needle, wherein when the drive component acts on the needle insertion tooth and the suture needle that are in an engagement state, the suture needle is controlled to be inserted, and the return component undergoes deformation when the suture needle is inserted; when the drive component is released, the engagement state of the needle insertion tooth and the suture needle is released, and the return component drives the needle insertion tooth to return; or when the drive component acts on the needle insertion tooth that is in a released engagement state, the needle insertion tooth moves relative to the suture needle, and the return component undergoes deformation when the needle insertion tooth moves relative to the suture needle; when the drive component is released, the needle insertion tooth engages with the suture needle, and the return component drives the needle insertion tooth and the suture needle to insert; and an anti-retraction component disposed within the main housing, the anti-retraction component forming a separable engagement relationship with the suture needle to prevent the suture needle from retracting.

One or more embodiments of the present disclosure provide a therapeutic apparatus, the therapeutic apparatus being used in conjunction with an endoscope, wherein the therapeutic apparatus includes: a main housing; a suture needle slidingly disposed within the main housing, wherein one end of the suture needle connects to a suture thread and the other end of the suture needle is a tip; a needle insertion assembly disposed within the main housing, the needle insertion assembly including a drive component, a return component, and a needle insertion tooth, the needle insertion tooth being in a separable engagement relationship with the suture needle, wherein when the drive component acts on the needle insertion tooth and the suture needle that are in an engagement state, the suture needle is controlled to be inserted, and the return component undergoes deformation when the suture needle is inserted; when the drive component is released, the engagement state of the needle insertion tooth and the suture needle is released, and the return component drives the needle insertion tooth to return; or the drive component acts on the needle insertion tooth that is in a released engagement state to make the needle insertion tooth move relative to the suture needle, and the return component undergoes deformation when the needle insertion tooth moves relative to the suture needle; when the drive component is released, the needle insertion tooth engages with the suture needle, and the return component drives the needle insertion tooth and the suture needle to insert; an anti-retraction component disposed within the main housing, the anti-retraction component forming a separable engagement relationship with the suture needle to prevent the suture needle from retracting; and a control handle fixed at an operating end of the endoscope, wherein the drive component of the suturing device is connected to the control handle, and the control handle controls the drive component to control the insertion of the suture needle.

One or more embodiments of the present disclosure provide a therapeutic device, comprising: an endoscope; a therapeutic apparatus used in conjunction with the endoscope, the therapeutic apparatus includes a suturing device and a control handle; the suturing device is fixed to an apex of the endoscope, and the suturing device includes: a main housing; a suture needle slidingly disposed within the main housing, wherein one end of the suture needle connects to a suture thread and the other end of the suture needle is a tip; a needle insertion assembly disposed within the main housing, the needle insertion assembly including a drive component, a return component, and a needle insertion tooth, the needle insertion tooth being in a separable engagement relationship with the suture needle, wherein when the drive component acts on the needle insertion tooth and the suture needle that are in an engagement state, the suture needle is controlled to be inserted, and the return component undergoes deformation when the suture needle is inserted; when the drive component is released, the engagement state of the needle insertion tooth and the suture needle is released, and the return component drives the needle insertion tooth to return; or the drive component acts on the needle insertion tooth that is in a released engagement state to make the needle insertion tooth move relative to the suture needle, and the return component undergoes deformation when the needle insertion tooth moves relative to the suture needle; when the drive component is released, the needle insertion tooth engages with the suture needle, and the return component drives the needle insertion tooth and the suture needle to insert; and an anti-retraction component disposed within the main housing, the anti-retraction component forming a separable engagement relationship with the suture needle to prevent the suture needle from retracting; and a control handle fixed at an operating end of the endoscope, wherein the drive component of the suturing device is connected to the control handle, and the control handle controls the drive component to control the insertion of the suture needle.

In order to make the above and other purposes, features, and advantages of the present disclosure more apparent and understandable, the following better embodiments are specially cited with the accompanying drawings for a detailed description of the present disclosure.

### BRIEF DESCRIPTION OF THE DRAWINGS

FIG. 1A is a schematic diagram illustrating a structure of a suturing device according to some embodiments of the present disclosure;

FIG. 1B and FIG. 1C are schematic diagrams illustrating a structure of a suturing device in other views according to some embodiments of the present disclosure;

FIG. 2 is a disassembled schematic diagram of a suturing device according to some embodiments of the present disclosure;

FIG. 3 is a schematic diagram illustrating the assembly of a suture needle and a needle insertion assembly in the suturing device according to some embodiments of the present disclosure;

FIG. 4A is a schematic diagram illustrating a structure of a suture needle according to some embodiments of the present disclosure;

FIG. 4B is a schematic diagram illustrating a structure of the suture needle in another view according to some embodiments of the present disclosure;

FIG. 4C shows a side view of a suture needle according to some embodiments of the present disclosure;

FIG. 5A to FIG. 5F show schematic diagrams of different states when an anti-retraction component, a suture needle, and a needle insertion assembly of a suturing device are in motion according to some embodiments of the present disclosure;

FIG. 6A shows a schematic diagram of a structure of a single cord (rope) control handle according to some embodiments of the present disclosure;

FIG. 6B to FIG. 6D are schematic diagrams illustrating a structure of a single cord (rope) control handle in other views according to some embodiments of the present disclosure;

FIG. 6E is a disassembled schematic diagram of a single cord (rope) control handle according to some embodiments of the present disclosure;

FIG. 7 is a schematic diagram illustrating the assembly of a manipulating rope pulley and a handle front housing on a single cord (rope) control handle according to some embodiments of the present disclosure;

FIG. 8 is a schematic diagram illustrating a structure of a manipulating rope connected to the manipulating rope pulley using a circular pin and a pin spring according to some embodiments of the present disclosure;

FIG. 9 is a schematic diagram illustrating the assembly of a manipulating rope, a circular pin, and a pin spring according to some embodiments of the present disclosure;

FIG. 10A is a schematic diagram illustrating the assembly of a pull rod, a handle rear housing, a clutch disk, and a manipulating rope pulley of a control handle according to some embodiments of the present disclosure;

FIG. 10B is a schematic diagram illustrating a structure of a pull rod, a handle rear housing, a clutch disk, and a manipulating rope pulley of a control handle in another view according to some embodiments of the present disclosure;

FIG. 10C is a disassembled schematic diagram of a pull rod, a handle rear housing, a clutch disk, and a manipulating rope pulley of a control handle according to some embodiments of the present disclosure;

FIG. 11A is a schematic diagram illustrating the assembly of a pull rod, a handle front housing, and a clutch disk of a control handle according to some embodiments of the present disclosure;

FIG. 11B is a schematic diagram illustrating a structure of a pull rod, a handle front housing, and a clutch disk of a control handle in another view according to some embodiments of the present disclosure;

FIG. 12 is a schematic diagram illustrating the installation of a connecting ring with an endoscope and a push catheter according to some embodiments of the present disclosure;

FIG. 13A is a schematic diagram illustrating a structure of a suturing device according to some other embodiments of the present disclosure;

FIG. 13B and FIG. 13C are schematic diagrams illustrating a structure of a suturing device in other views according to some other embodiments of the present disclosure;

FIG. 14 shows a disassembled schematic diagram of a suturing device according to some other embodiments of the present disclosure;

FIG. 15 is a schematic diagram illustrating the assembly of a suture needle and a needle insertion assembly in a suturing device according to some other embodiments of the present disclosure;

FIG. 16A to FIG. 16F show schematic diagrams of different states when an anti-retraction component, a suture needle, and a needle insertion assembly of a suturing device are in motion according to some other embodiments of the present disclosure;

FIG. 17A is a schematic diagram illustrating a structure of a suturing device according to some other embodiments of the present disclosure;

FIG. 17B and FIG. 17C are schematic diagrams illustrating a structure of a suturing device in other views according to some other embodiments of the present disclosure;

FIG. 18 shows a disassembled schematic diagram of a suturing device according to some other embodiments of the present disclosure;

FIG. 19A is a schematic diagram illustrating the assembly of a second housing, a third housing, and an external clamp catheter of a suturing device according to some other embodiments of the present disclosure;

FIG. 19B is a schematic diagram illustrating a structure of a second housing, a third housing, and an external clamp catheter of the suturing device in another view according to some other embodiments of the present disclosure;

FIG. 20 shows a partially enlarged view of a second housing, a third housing, and an external clamp catheter of the suturing device according to some other embodiments of the present disclosure; and

FIG. 21A, FIG. 21B, and FIG. 21C are schematic diagrams of adjustable tubular portions according to some embodiments of the present disclosure.

Descriptions of figure markings: 1: suturing device, 11: main housing, 111: first housing, 1111: first channel, 1112: second channel, 1113: anti-tangle sliding column, 112: second housing, 1121: sliding groove, 12: needle insertion assembly, 121: drive component, 122: return component, 123: needle insertion tooth, 124: slider, 1240: holding groove, 1241: slider body, 1242: slider connecting portion, 125: upper housing, 1251: holding cavity, 126: upper cover, 127: coupling block, 128: needle insertion tooth shrapnel, 129: needle insertion tooth spring, 13: suture needle, 131: suture thread, 132: slot, 14: anti-retraction component, 141: lower housing, 1411: positioning hole, 142: anti-retraction tooth, 143: anti-retraction tooth shrapnel, 2: control handle, 21: handle housing, 211: front housing, 212: middle housing, 213: rear housing, 22: manipulating rope pulley, 221: side hole, 222: viewing hole, 223: latch sliding groove, 224: slot ring, 23: clutch disk, 231: snap ring, 24: pull rod, 25: tension spring, 26: compression spring, 27: manipulating rope retainer, 271: circular pin, 272: pin spring, 28: guide cylindrical pin, 200: endoscope, 201: operating end of the endoscope, 202: apex of the endoscope, 3: external clamp catheter, 4: connecting ring, 5: push catheter, 113′: third housing, 114′: rotating gear, 1114′: locating component mounting portion, 31′: clamp catheter body, 32′: clamp catheter drive tooth, 321′: circumferential locating hole, 33′: locating component, 331′: locating pin, 332′: locating pin spring, 333′: locating pin pull cope, 34′: protective cover.

### DETAILED DESCRIPTION

This present disclosure provides a suturing device, a single cord (rope) control handle coupled with the suturing device, a therapeutic apparatus consisting of the suturing device and the control handle, and a therapeutic device consisting of the therapeutic apparatus and an endoscope coupled with the therapeutic apparatus. The suturing device may be used to perform in vivo sutures on mammalian tissues, whether or not the subject is a human, and whether or not the subject has vital signs. The suturing device provided herein may be used not only with the endoscope but also with other devices capable of steering guidance.

In some embodiments, the suturing device includes a main housing, a suture needle, a needle insertion assembly, and an anti-retraction component, etc.

The suture needle is configured to suture a target suturing site of an object to be sutured. The target suturing site may be an area or tissue to be sutured, etc. In some embodiments, the suture needle may be slidably disposed within the main housing. One end of the suture needle is connected to a suture thread, and the other end of the suture needle is a tip.

The needle insertion assembly is configured to control needle insertion and return of the suture needle, etc. In some embodiments, the needle insertion assembly is disposed within the main housing. The needle insertion assembly includes a drive component, a return component, a needle insertion tooth, etc.

The drive component is configured to drive the insertion of the suture needle and the needle insertion tooth, etc. The return component is configured to drive the needle insertion tooth to return. The needle insertion tooth is configured to snap the suture needle.

In some embodiments, the needle insertion tooth is in a separable engagement relationship with the suture needle. The drive component acts on the needle insertion tooth and the suture needle in an engagement state to control insertion of the suture needle. The return component undergoes deformation when the suture needle is inserted. When the drive component is released, the engagement state of the needle insertion tooth and the suture needle is released, and the return component drives the needle insertion tooth to return.

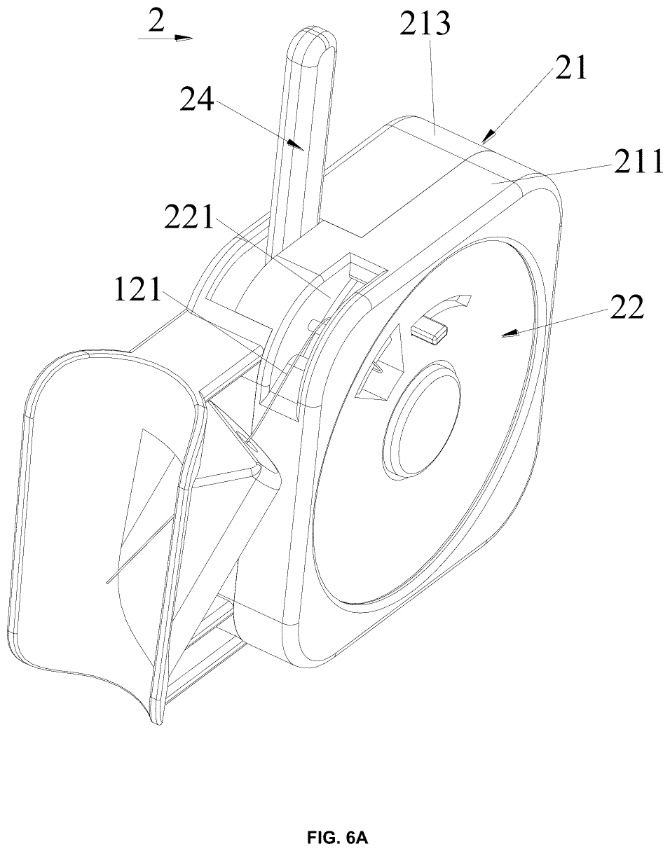

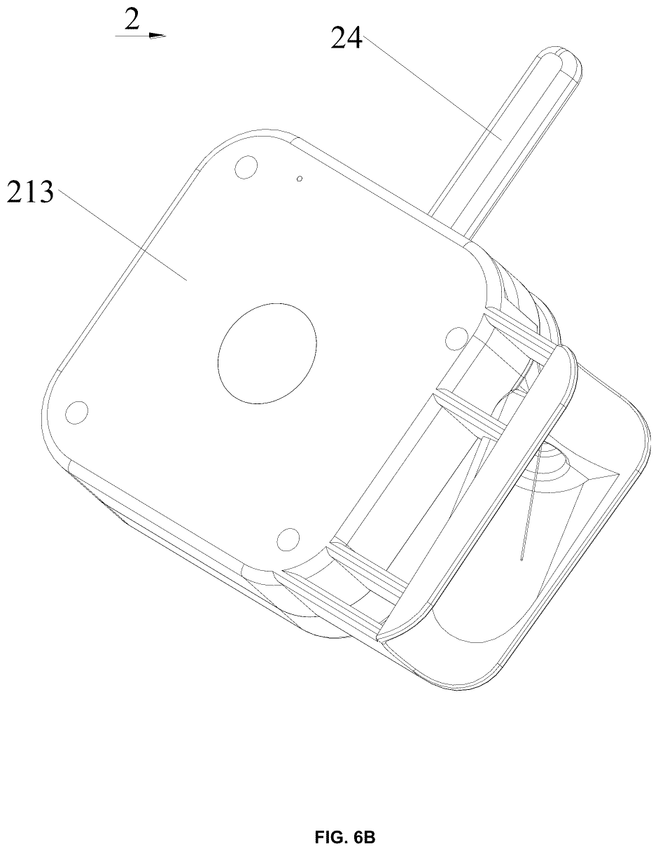

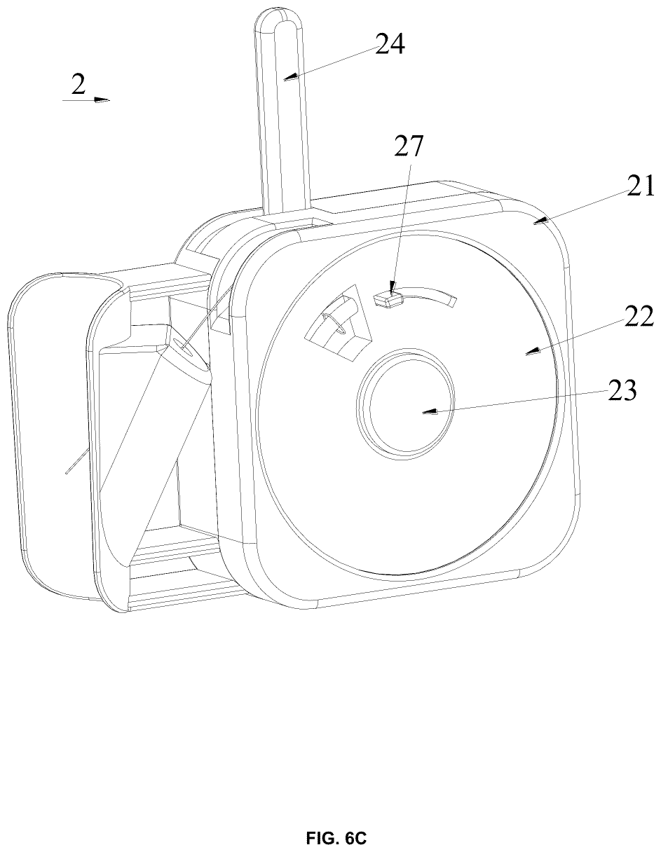

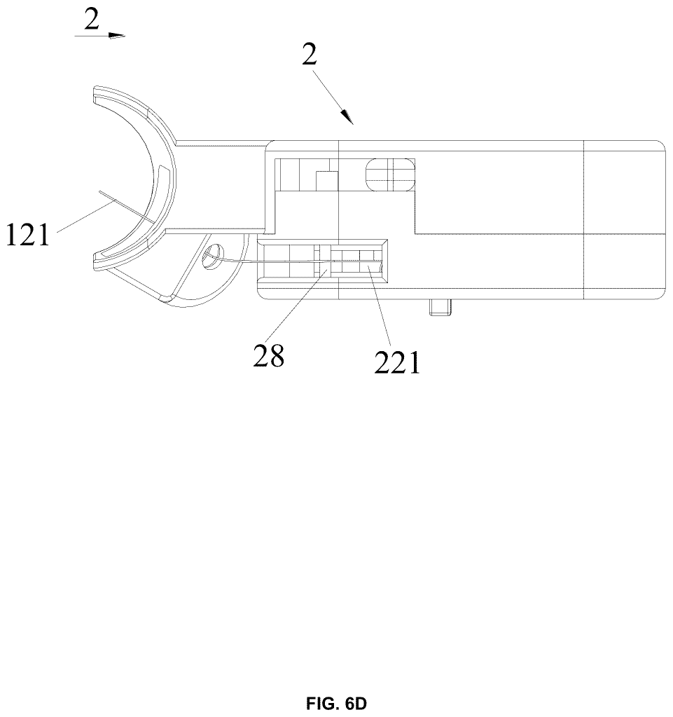

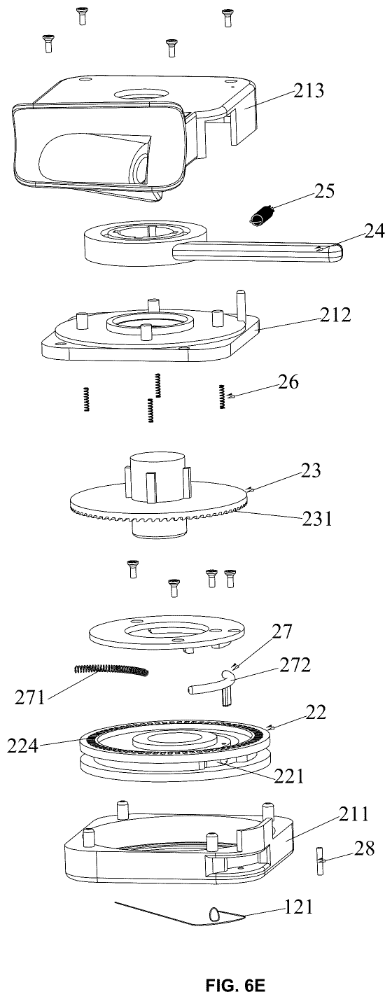

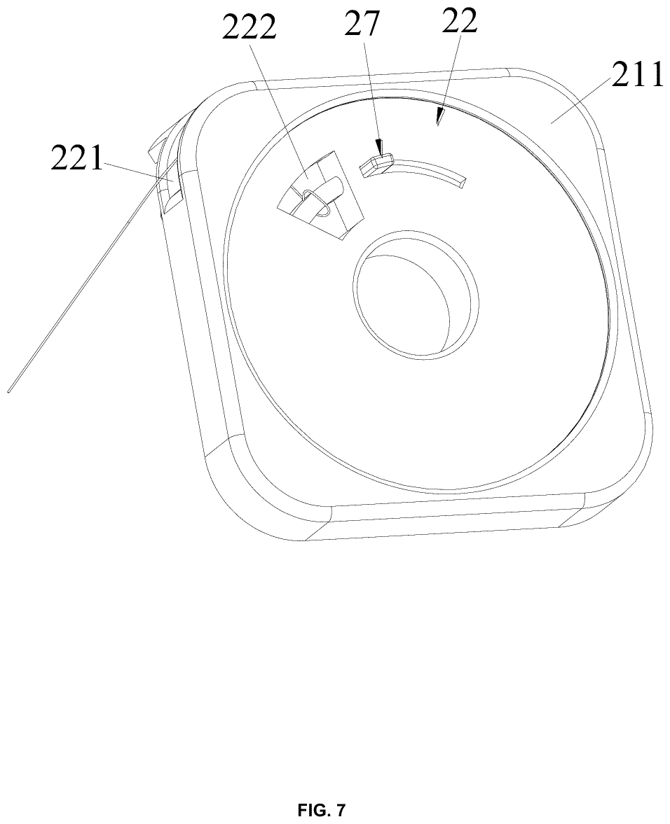

The drive component being released refers that the drive component does not produce an output of force. In some embodiments, the release of the drive component may be accomplished by the control handle. More descriptions regarding the control handle may be found in FIG. 6A to FIG. 11B and the relevant descriptions.

The separable engagement relationship refers to an engagement relationship that may be formed or dissolved.

In other embodiments, the drive component acts on the needle insertion tooth that is in a released engagement state, the needle insertion tooth moves relative to the suture needle, and the return component undergoes deformation when the needle insertion tooth moves relative to the suture needle. When the drive component is released, the needle insertion tooth engages with the suture needle, and the return component drives the needle insertion tooth and the suture needle to insert.

The anti-retraction component is configured to prevent the suture needle from retracting. In some embodiments, the anti-retraction component is disposed within the main housing, and the anti-retraction component forms a separable engagement relationship with the suture needle to prevent the suture needle from retracting.

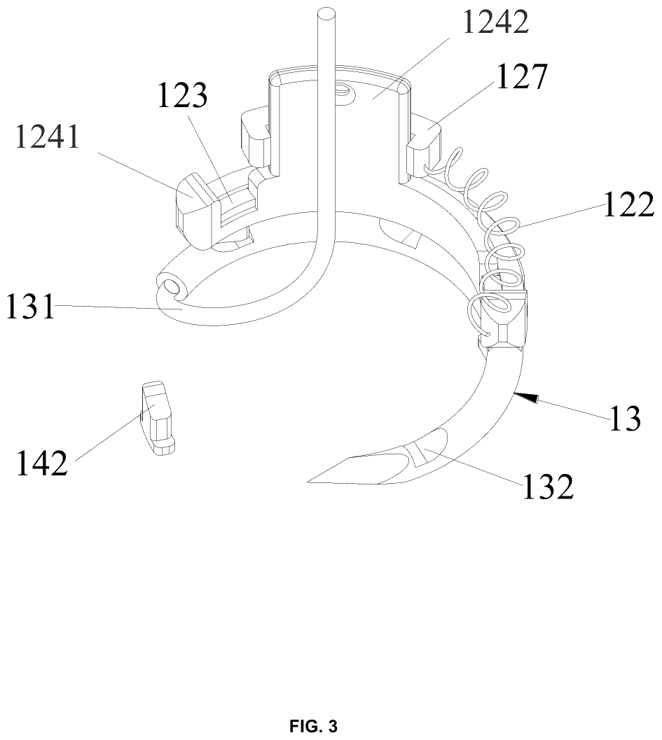

In some embodiments, the drive component includes a manipulating rope that acts on the needle insertion tooth. The return component includes at least one of a return spring or a metallic spring plate that acts on the needle insertion tooth. An operator (e.g., a doctor, etc.) applies force to the needle insertion tooth by pulling the manipulating rope and stops applying force to the needle insertion tooth by relaxing the manipulating rope. More descriptions regarding the drive component and the return component may be found in FIG. 3 and the relevant descriptions.

In some embodiments, the main housing has a sliding groove, and the suture needle is disposed within the sliding groove. The needle insertion assembly further includes a slider disposed within the sliding groove and above the suture needle. The needle insertion tooth is retractably assembled in the slider, and the drive component and the return component are both connected to the slider. The slider may be used to drive the needle insertion tooth to move.

The needle insertion tooth being retractably assembled in the slider refers that the needle insertion tooth is assembled in the slider and contracted in the slider when subjected to force, and extended outside the slider when not subjected to force.

In some embodiments, the needle insertion assembly further includes an upper housing that covers the main housing and an upper cover that covers the upper housing, etc., and a holding cavity is formed between the upper housing and the upper cover.

In some embodiments, the slider includes a slider body and a slider connecting portion, etc., and the slider connecting portion sequentially passes through the upper housing and the upper cover. The return component is disposed within the holding cavity, one end of the return component is connected to the slider connecting portion, the other end of the return component is connected to the upper housing or the upper cover, and the drive component is connected to an upper end of the slider connecting portion.

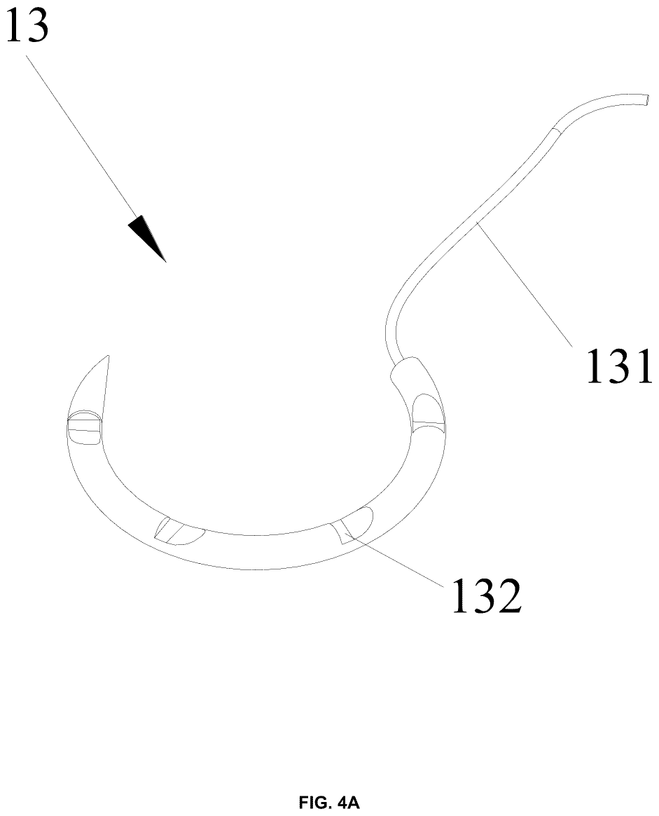

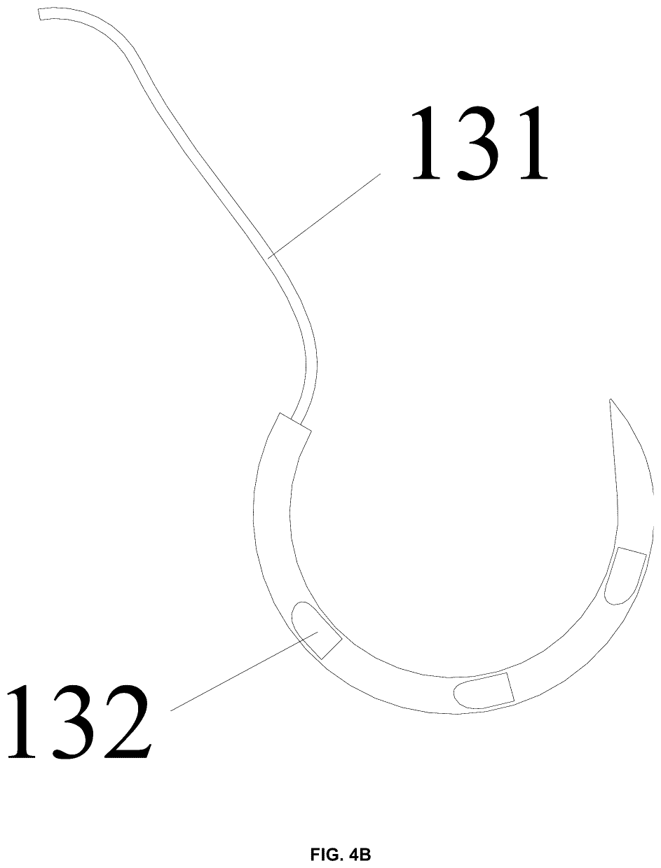

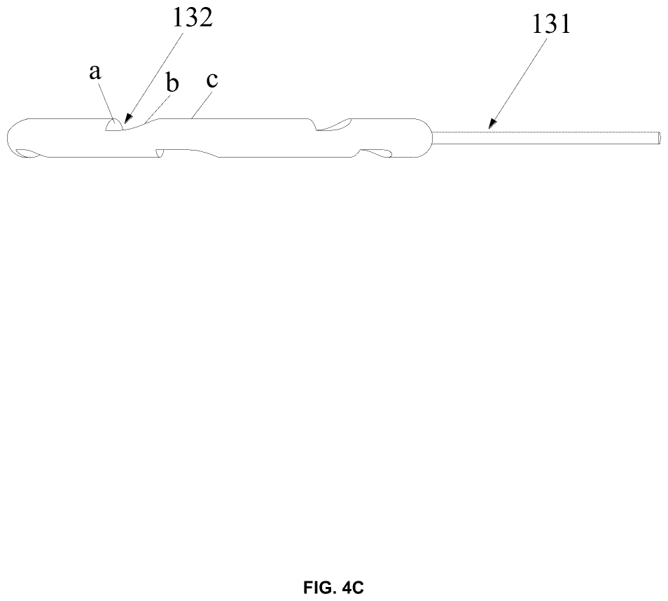

The slider connecting portion may be used to connect the return component, the slider body, and the drive component. More descriptions regarding the slider may be found in FIG. 4 and the relevant descriptions.

In some embodiments, the bottom of the slider has a holding groove. The return component is disposed in the holding groove and is confined between the slider and the suture needle. One end of the return component is connected to a side wall of the holding groove, and the other end of the return component is connected to an inner wall of the sliding groove of the main housing.

In some embodiments, the suturing device further includes an external clamp catheter. The main housing includes a first housing, a second housing, a third housing, etc. The first housing is connected to the second housing, the third housing and the second housing are rotated relative to the first housing. The external clamp catheter is drivably connected to the first housing to drive relative rotation among the first housing, the second housing, and the third housing, so as to arbitrarily convert an orientation and a position of a C-shaped opening of the suturing device.

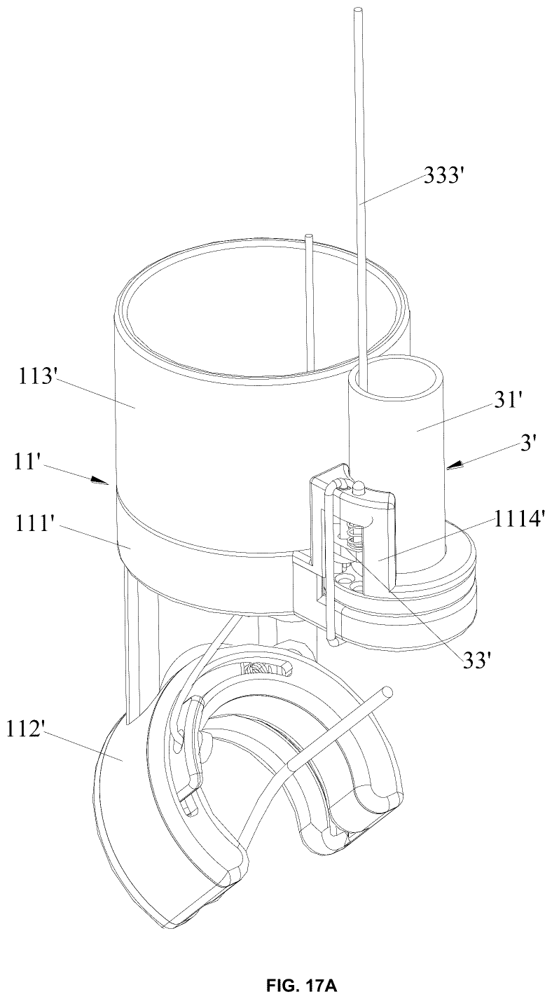

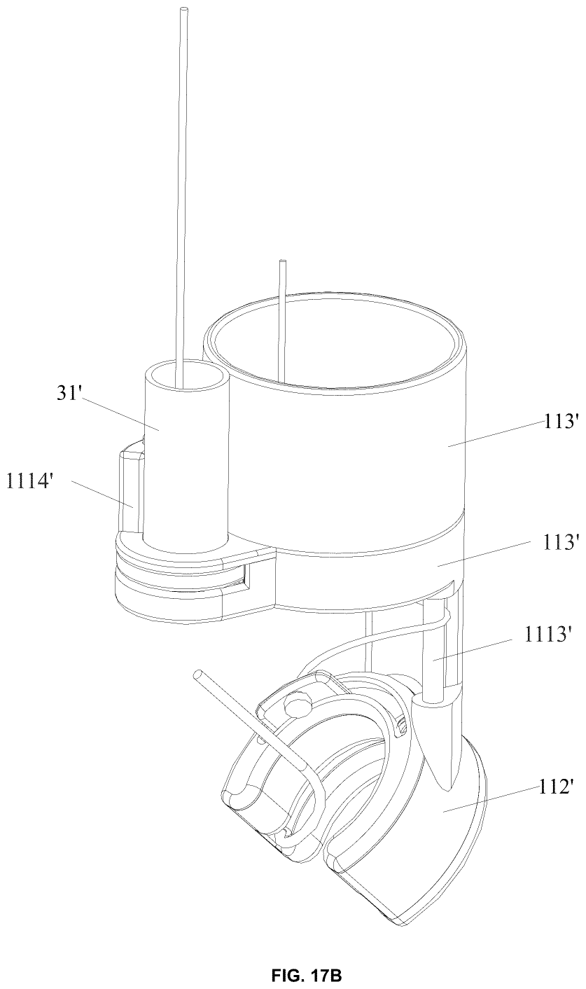

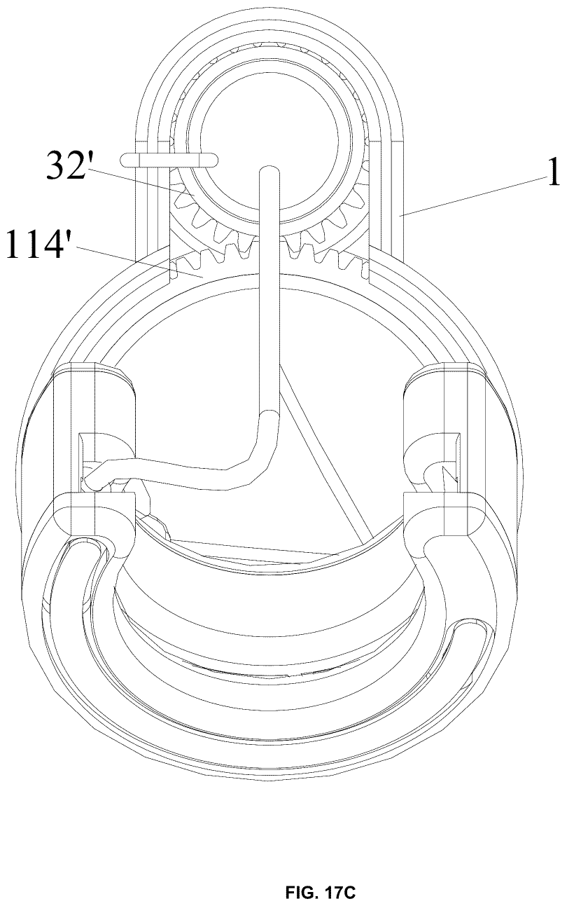

In other embodiments, the first housing may also be rotationally connected to the third housing via a rotating gear of the main housing. The drive teeth of the external clamp catheter engage with the rotating gear, and the external clamp catheter drives the first housing and the second housing via the transmission of the clamp catheter drive tooth and the rotating gear. This setting facilitates coaxial rotation of the suturing device in a direction pointed by the C-shaped opening in the body, especially in a narrow internal space. More descriptions regarding the external clamp catheter and the rotating gear may be found in FIG. 17A to FIG. 20 and the relevant descriptions.

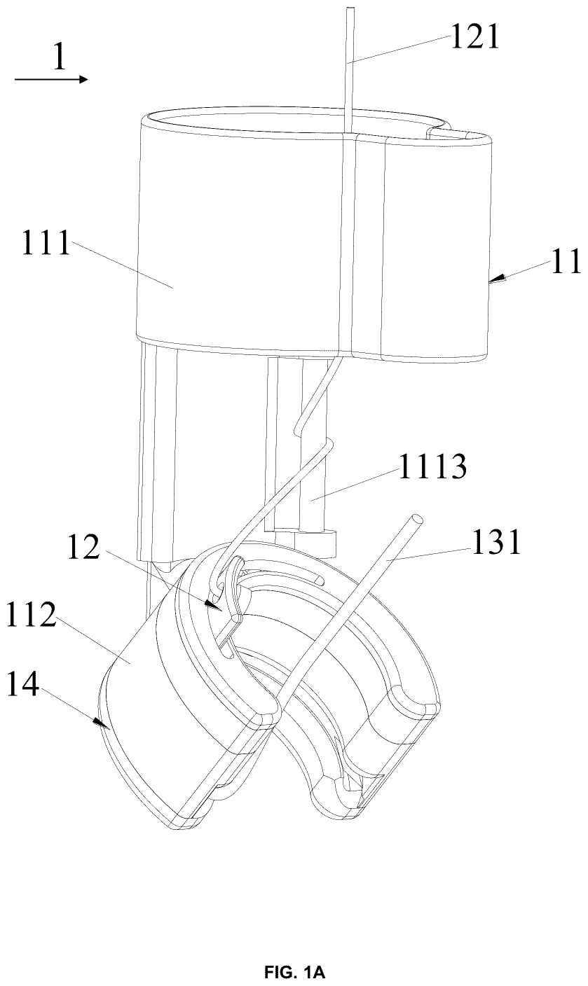

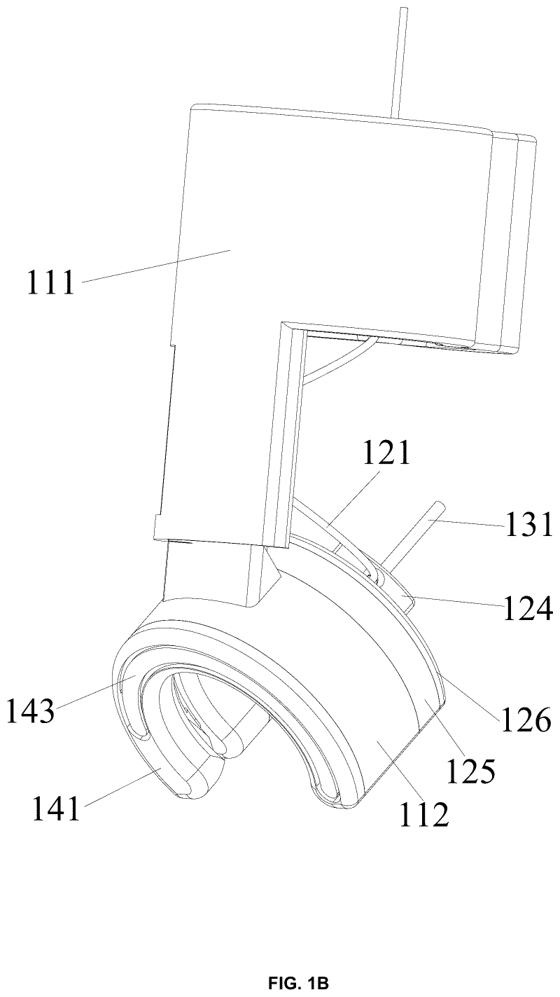

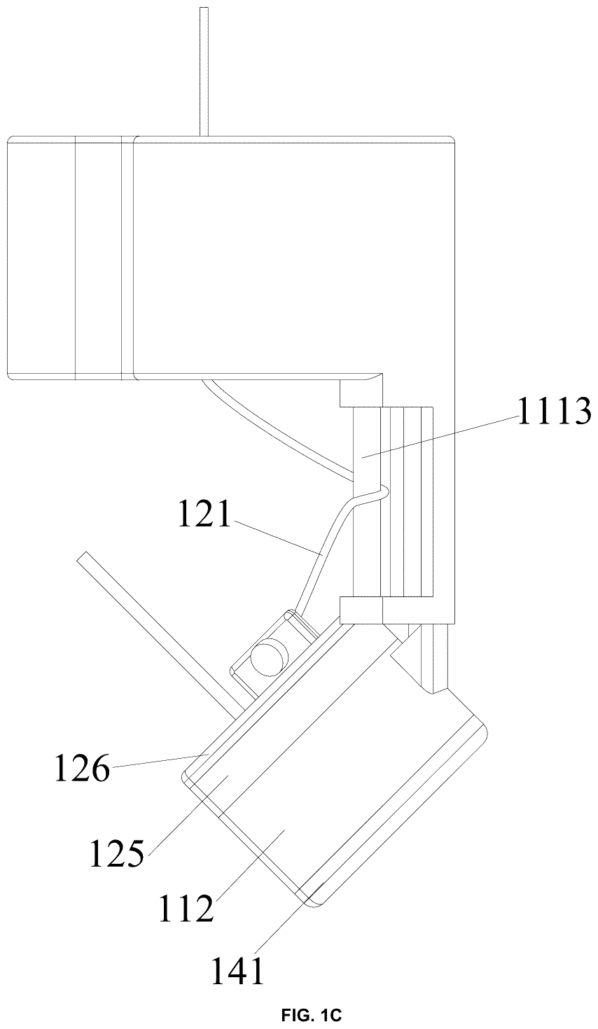

In some embodiments, the therapeutic apparatus provided herein includes a suturing device and a control handle (e.g., a single cord (rope) control handle, etc.). The suturing device is fixed to the apex of the endoscope, and the therapeutic apparatus is used in conjunction with the endoscope. More descriptions regarding the suturing device may be found in FIG. 1A to FIG. 3 and the relevant descriptions.

In some embodiments, the suturing device is fixed to the apex of the endoscope. The control handle is fixed to the operating end of the endoscope. The drive component of the suturing device is connected to the control handle, and the control handle controls the drive component to control the insertion of the suture needle.

In some embodiments, the control handle includes a handle housing, a manipulating rope pulley, a clutch disk, and a pull rod.

In some embodiments, the manipulating rope pulley is provided within the handle housing. The drive component on the suturing device is a manipulating rope, and the manipulating rope is connected to the manipulating rope pulley.

In some embodiments, the clutch disk is detachably disposed on a side of the manipulating rope pulley, and the clutch disk is provided with a snap ring on the side proximate to the manipulating rope pulley that engages the manipulating rope pulley. The pull rod is attached to the clutch disk and resettably attached to the handle housing. More descriptions regarding the control handle may be found in FIG. 6A to FIG. 11B and the relevant descriptions.

In some embodiments of the present disclosure, the control handle utilizes the engagement between a small-sized clutch disk and the manipulating rope pulley to achieve an increase in rotational torque, which achieves miniaturization of the control handle and reduces cost. Using a curved side wall of the manipulating rope to pull the manipulating rope greatly reduces the resistance of the manipulating rope to move, not only is the operation of the pull rod easier, but the operator has good compliance and it also greatly reduces the damage of the manipulating rope to various parts of the suturing device, extending the service life of the suturing device. The control handle shortens a distance between the pull rod and the base of the thumb of the operator, reduces the range of finger spreading and hooking, which is in line with the ergonomics of the hand and is not likely to induce damage to the operator's hand tendon sheath, ligament, or muscle, making the operation more relaxing, suturing more accurate, surgery more rapid, and the connection of the suture more lasting, and the finger fatigue slower.

In some embodiments, the therapeutic apparatus further includes a connecting ring, a loop ring, a hook clamp, and a push catheter. The connecting ring is fitted onto the endoscope, the loop ring performs a cutting action of the target suturing tissue (also referred to as the target suturing site) at the apex of the endoscope, and the hook clamp performs a grabbing action on the target suturing tissue or a hooking and pulling action on the suture thread at the apex of the endoscope. The push catheter is configured to push forward longitudinally and assist in tightening the knot horizontally.

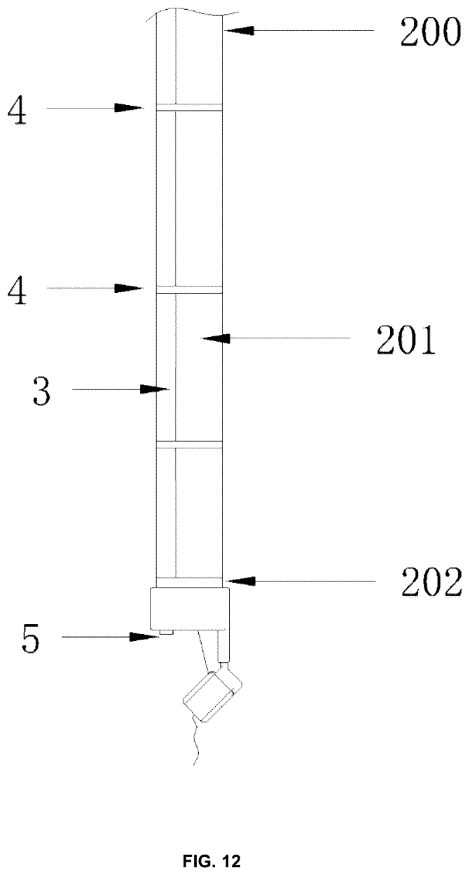

More descriptions regarding the connecting ring, the loop ring, the hook clamp, and the push catheter may be found in FIG. 12 and the relevant descriptions.

In some embodiments, the therapeutic device provided herein includes an endoscope and a therapeutic apparatus. The therapeutic apparatus is used in conjunction with the endoscope. The therapeutic apparatus includes a suturing device and a control handle. More descriptions regarding the suturing device may be found in FIG. 1A to FIG. 3 and the relevant descriptions. More descriptions regarding the control handle may be found in FIG. 6A to FIG. 11B and the relevant descriptions.

In some embodiments of the present disclosure, in the suturing device, and the therapeutic device and the therapeutic device having the suturing device provided herein, the needle insertion assembly includes the drive component, the return component, and the needle insertion tooth. When the drive component drives the needle insertion tooth to move, the return component synchronously deforms. When the drive component is released, a return force of the return component drives the needle insertion tooth to return. This arrangement allows the insertion of the suture needle to be achieved solely by controlling the drive component through a single cord (rope) control handle. During the procedure, the operator of the endoscope only needs to control the control handle with one hand, while the other hand can continue to suture. The operation of a single component also allows the operator to control the control handle without sight, ensuring that the operator's attention is always focused on the operation of the endoscope, thereby ensuring the safety and continuity of the surgical operations.

In some embodiments of the present disclosure, the starting needle insertion of a single thread makes the suturing device simple in shape, reduces resistance during operation, does not obstruct the field of view when stitching, is not easy to be entangled, and also can be rotated coaxially with the suturing device to accurately reach the target suturing site, achieving the best suturing result. Using the suturing device of the present disclosure and adopting cross suturing, there is no limitation on the size of the wound or the size of the target suturing tissue.

More descriptions regarding the suturing device, the control handle, the therapeutic devices, and the therapeutic apparatus, etc., may be found in FIG. 1A to FIG. 21C and the relevant descriptions.

FIG. 1A is a schematic diagram of a structure of a suturing device provided in some embodiments of the present disclosure.

FIG. 1B and FIG. 1C are schematic diagrams illustrating a structure of a suturing device in other views according to some embodiments of the present disclosure.

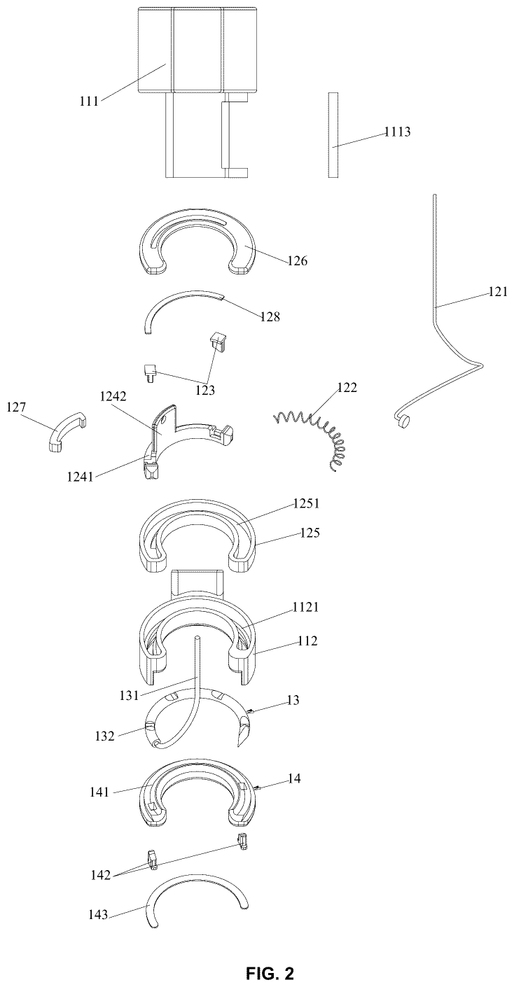

FIG. 2 is a disassembled schematic diagram of a suturing device according to some embodiments of the present disclosure.

### Example 1

In some embodiments, as shown in FIG. 1A to FIG. 2, the present disclosure provides a suturing device 1 for puncturing and suturing tissues to perform surgical procedures in the body. The suturing device 1 includes a main housing 11, a needle insertion assembly 12, a suture needle 13, and an anti-retraction component 14.

In some embodiments, the suturing device 1 is used in conjunction with an endoscope, and the main housing 11 includes a first housing 111 and a second housing 112. The endoscope (not shown in the figure) is connected to the first housing 111. The first housing 111 has a variable diameter and may be constituted by any one or a combination of self-locking clasp teeth, chains, and/or pins and bolts, etc., so as to adapt to the apex of the endoscopes installed on the first housing 111 of different diameters. The disclosure does not make any limitation on the form of the constitution of the first housing.

In some embodiments, the needle insertion assembly 12, the suture needle 13, and the anti-retraction component 14 are provided on the second housing 112.

In some embodiments of the present disclosure, the integrally molded main housing 11 makes the connection between the first housing 111 and the second housing 112 to be in a smooth transition, and the connection is free of edges and corners, which greatly reduces the damage caused by the contact of the suturing device with the internal tissue.

The present disclosure does not limit the manner in which the first housing and the second housing are connected. In other embodiments, the first housing and the second housing may also be connected in a split manner by a connector.

In some embodiments, when the suturing device is used in conjunction with an endoscope with a single clamp channel, the center of the first housing 111 may have a first channel and a second channel, with the first channel being snapped onto the apex of the endoscope, and the second channel being available for the passage of an external clamp catheter to secure the external clamp catheter. The present disclosure does not limit the setting of the first channel and the second channel. More descriptions regarding the external clamp catheter may be found in FIG. 12 and the relevant descriptions.

In some embodiments, when the suturing device is used in conjunction with an endoscope with a dual clamp channel, the first housing may have only one channel, allowing the suturing device to be snapped onto the apex of the endoscope. In other embodiments, the first housing may also be free from having a channel, but instead be fixed to the apex of the endoscope by other structures such as retaining straps, retaining rings, or the like, thereby enabling the suturing device to be fixed to the apex of the endoscope.

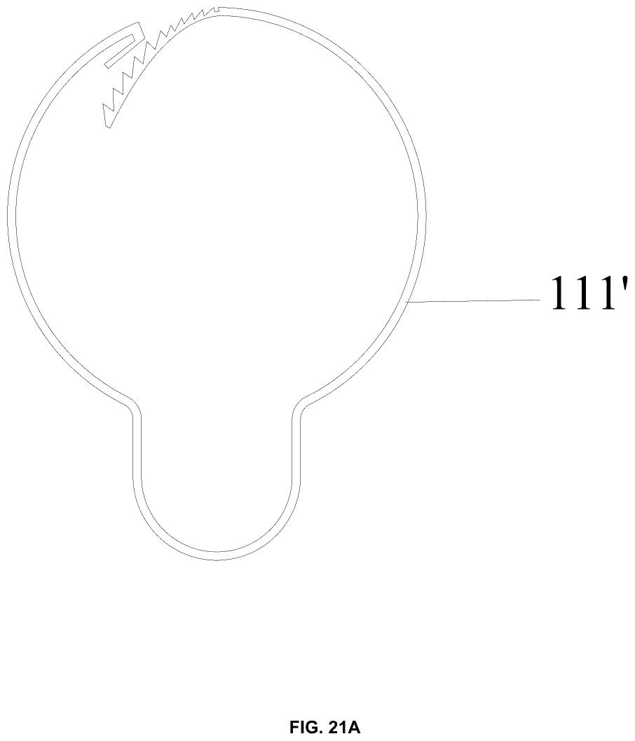

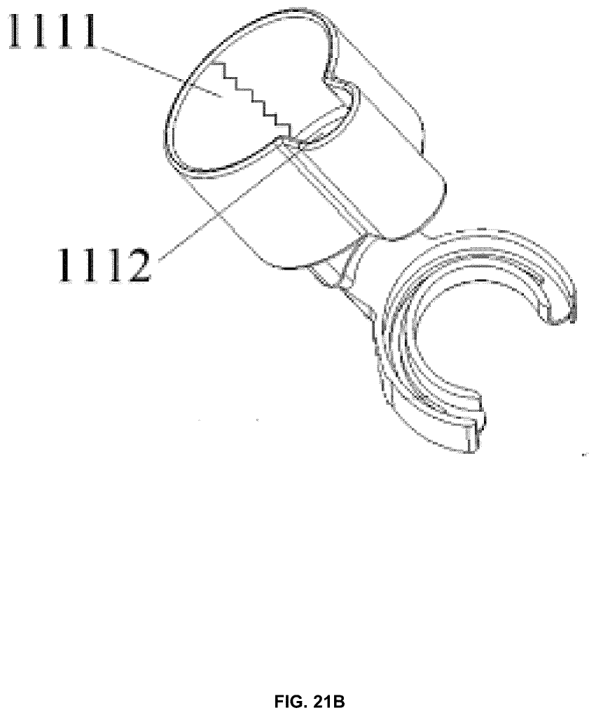

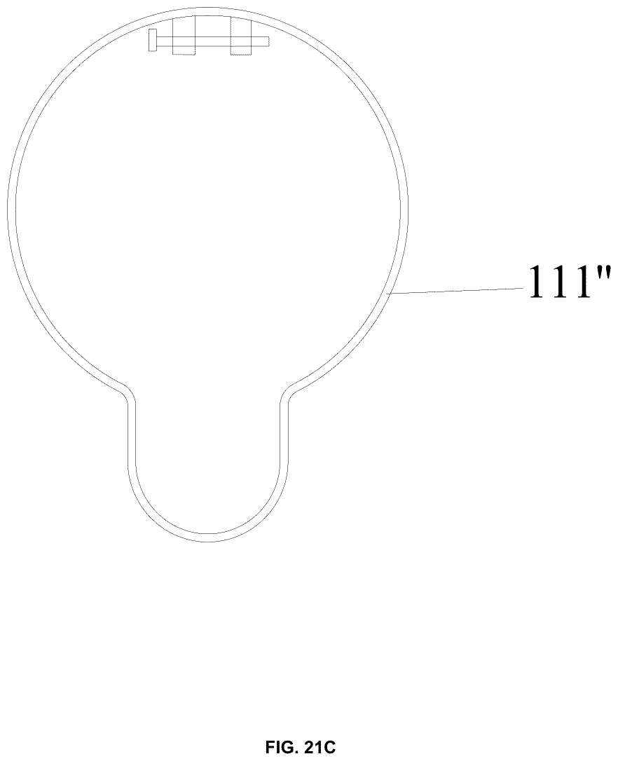

FIG. 21A, FIG. 21B, and FIG. 21C are schematic diagrams of adjustable tubular portions according to some embodiments of the present disclosure.

In some embodiments, the first housing may be a tubular structure with an adjustable diameter, also referred to as an adjustable tubular portion.

In some embodiments, as shown in FIG. 21A, FIG. 21B, and FIG. 21C, when the suturing device is used in conjunction with the endoscope having a single clamp channel, the middle of the first housing may have a first channel 1111 and a second channel 1112, and the adjustable tubular portion may be the first channel with an adjustable diameter.

In some embodiments, the adjustable tubular portion may include a plurality of structures. For example, the adjustable tubular portion (e.g., 111′ of FIG. 21A) is provided with a stepped snap structure on one side of a longitudinal edge of the adjustable tubular portion, and the other side of the longitudinal edge of the adjustable tubular portion is provided with a latch corresponding to the snap structure. The latch may be engaged with the snap structure when the snap structure is pushed towards one side of the latch. The greater the distance that the snap structure is pushed towards the side of the latch, the smaller the diameter of the adjustable tubular portion is.

As another example, the adjustable tubular portion (e.g., 111″ of FIG. 21C) is provided with a clamp at the longitudinal edge, where the diameter of the adjustable tubular portion is reduced by tightening bolts in the clamp and the diameter of the adjustable tubular portion is increased by loosening the bolts in the clamp.

In some embodiments, the first housing 111 also has an anti-tangle sliding column 1113 on an end of the first housing 111 proximate to the second housing 112, and a manipulating rope as a drive component 121 may pass through the anti-tangle sliding column 1113 and enter the first housing 111 from an inner side. The anti-tangle sliding column 1113 guides the relaxed manipulating rope between the first housing 111 and the second housing 112, avoiding tangling of the manipulating rope in the relaxed state. The present disclosure does not limit the setting of the anti-tangle sliding column.

In some embodiments, as shown in FIG. 2, a surface of the second housing 112 proximate to the first housing 111 has a sliding groove 1121. The suture needle 13 is provided within the sliding groove 1121. The needle insertion assembly 12 is provided within the sliding groove 1121.

In some embodiments, the needle insertion assembly 12 includes the drive component 121, a return component 122, and a needle insertion tooth 123. The needle insertion tooth 123 is in a separable engagement relationship with the suture needle 13.

FIG. 3 is a schematic diagram illustrating the assembly of a suture needle and a needle insertion assembly in the suturing device according to some embodiments of the present disclosure.

In some embodiments, referring to FIG. 3, during the first needle insertion, the drive component 121 acts on the needle insertion tooth 123 to cause the needle insertion tooth 123 to snap onto the suture needle 13, at which time the needle insertion tooth 123 and the suture needle 13 are in a coupled state (also referred to as an engagement state), which in turn controls the insertion of the suture needle 13, while the return component 122 undergoes deformation. When the drive component 122 is released, the engagement state of the needle insertion tooth 123 and the suture needle 13 is released, and the return component 122 drives the needle insertion tooth 123 to return.

At each subsequent needle insertion, the drive component 121 and the return component 122 repeat the process of the first needle insertion, realizing continuous suturing. The present disclosure does not limit the process of the action of the drive component and the return component in any way.

In other embodiments, it is also possible to adjust the snap structure of the needle insertion tooth and the suture needle so that the drive component, when acting on the needle insertion tooth, drives the needle insertion tooth, which are released from the engagement relationship, to move relative to the suture needle, and at the same time the return component deforms. When the drive component is released, the needle insertion tooth is engaged with the suture needle, and the return component drives the needle insertion tooth and the suture needle forward.

In the suturing device provided in some embodiments of the present disclosure, the insertion of the suture needle 13 can be realized by controlling the single component of the drive component 121, the operator can control it with only one hand, the other hand can maintain the continuous operation of the endoscope, and the operation is simpler and more convenient. At the same time, this setting also makes it possible for the second housing 112 to be provided with only one sliding groove 1121 to accommodate the suture needle 13 and the drive component 112 at the same time, which greatly simplifies the structure of the second housing 112, reduces the manufacturing cost, and at the same time shrinks the size of the suturing device.

In some embodiments, the drive component 121 includes a manipulating rope, or the like, and the return component 122 includes a return spring, or the like. The present disclosure does not limit the selection of the drive component and the return component in any way.

In other embodiments, the drive component 121 may also be free of the drawstring, and control of the suture needle may be achieved through other structural alternatives. The present disclosure does not limit this in any way. The return component may also be replaced by a structure made of a metallic spring plate or a polymer material, such as elastic silicone, etc., which has a certain restoring force or elasticity. The present disclosure does not limit this in any way.

In some embodiments, referring to FIG. 1A to FIG. 3, the needle insertion assembly 12 further includes a slider 124 disposed within the sliding groove 1121 and disposed above the suture needle 13, an upper housing 125 capped above the second housing 112, and an upper cover 126 capped above the upper housing 125. The needle insertion tooth 123 is retractably assembled to the slider 124, and the drive component 121 and the return component 122 are both connected to the slider 124. The holding cavity 1251 is formed between the upper housing 125 and the upper cover 126.

In some embodiments, the slider 124 includes a slider body 1241 and a slider connecting portion 1242, and the slider connecting portion 1242 passes through the upper housing 125 and the upper cover 126 in turn. The return component 122 is provided in the holding cavity 1251, one end of the return component 122 is connected to the slider connecting portion 1242, and the other end of the return component 122 is connected to the upper housing 125 or the upper cover 126. The drive component 121 is connected to an upper end of the slider connecting portion 1242.

In some embodiments of the present disclosure, the holding cavity 1251 formed by the upper housing 125 and the upper cover 126 limits the direction of the deformation of the return component 122, preventing the return component 122 from arching in other directions when the deformation occurs, which affects the direction of the restoring force. This setting ensures that the deformation of the return component 122 occurs in the circumferential direction of the sliding groove 1121, so that the slider 124 and the needle insertion tooth 123 can be smoothly driven to be return along the sliding groove 1121 during return, and thus driving the suture needle 13 to move forward stably. In some embodiments, the height of the holding cavity 1251 is provided to be greater than the diameter of the return component 122 (e.g., a return spring, etc.), and a difference between the height and the diameter is less than a predetermined difference. The predetermined difference is predetermined based on historical experience. The present disclosure does not limit the height of the holding cavity in any way.

In some embodiments, the return component 122 is detachably connected to the slider connecting portion 1242 via a coupling block 127 (e.g., the coupling block 127 snaps onto the slider connecting portion 1242, one end of the return component 122 is connected to the coupling block 127, etc.). The present disclosure does not limit the connection manner between the connecting block and the slider.

In other embodiments, the coupling block 127 may also be removably attached to the slider connecting portion using a connecting component such as a screw, etc.

In some embodiments, the needle insertion assembly 12 may include two or more needle insertion teeth. The present disclosure does not limit a count of needle insertion teeth in any way.

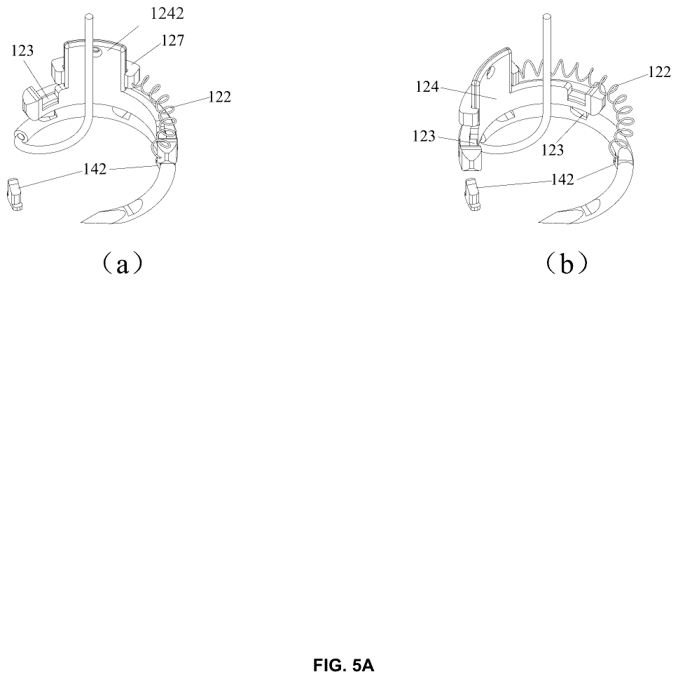

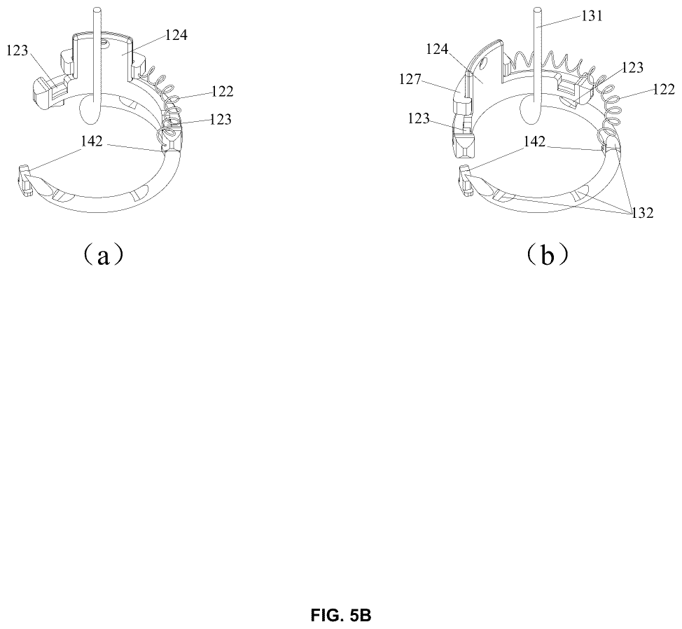

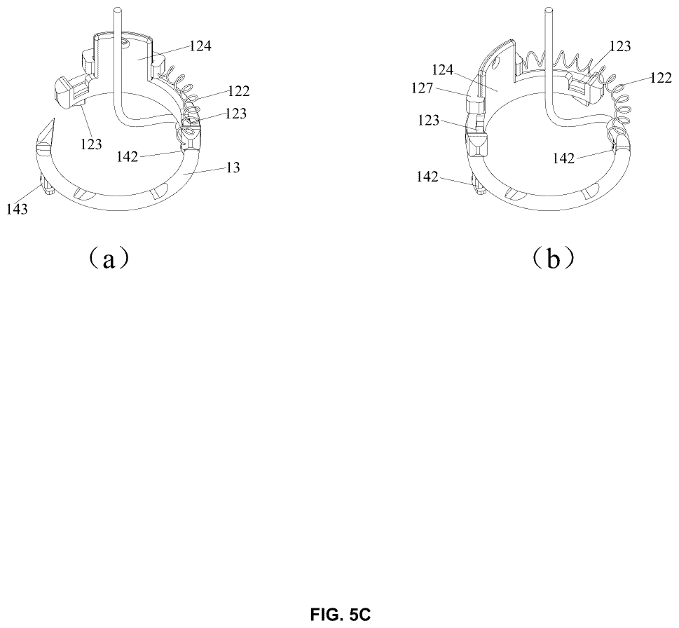

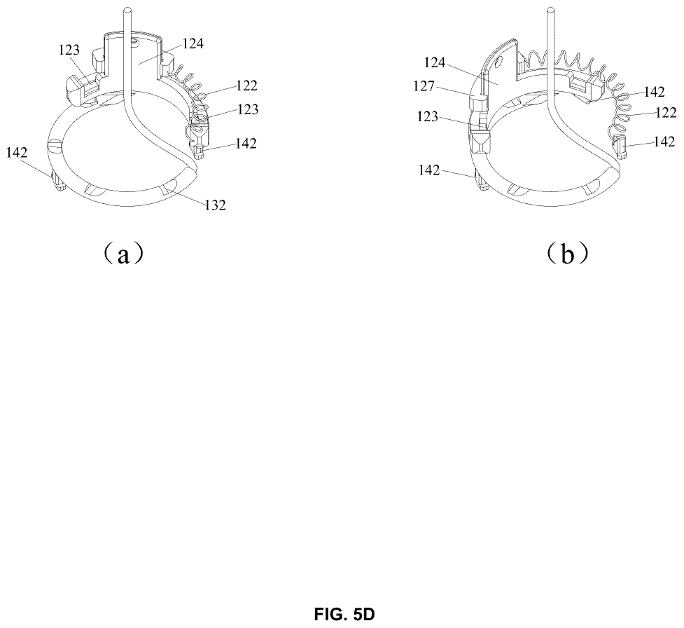

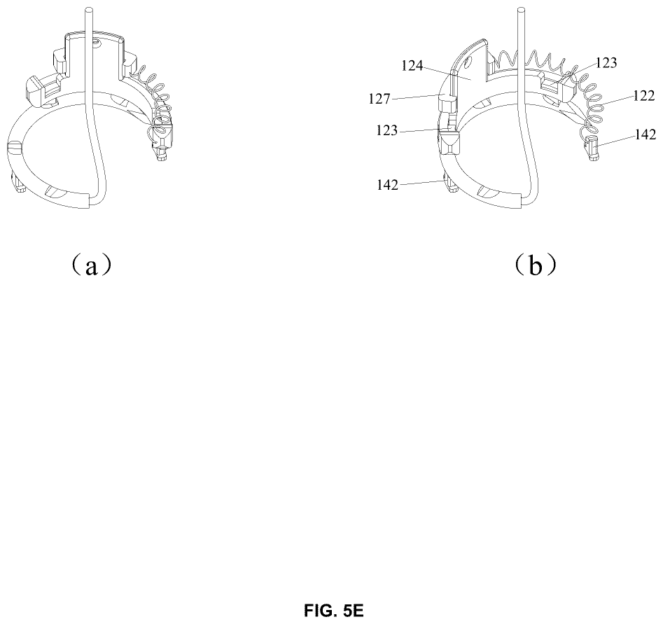

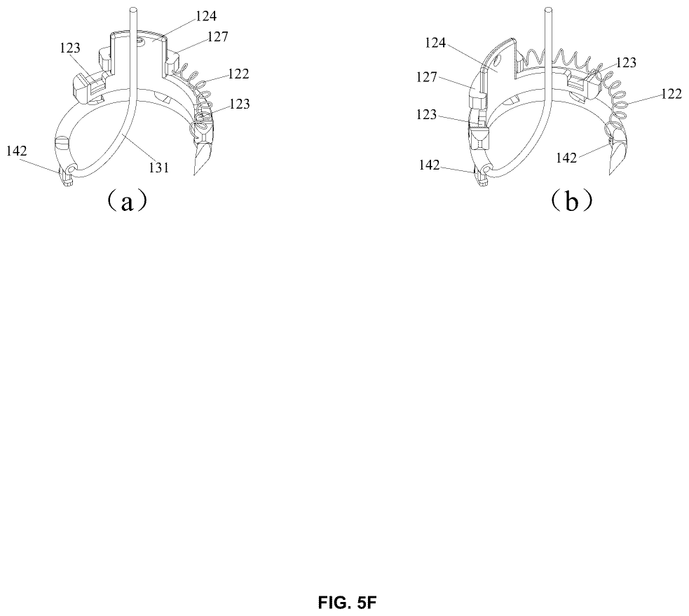

FIG. 5A to FIG. 5F show schematic diagrams of different states when an anti-retraction component, a suture needle, and a needle insertion assembly of a suturing device are in motion according to some embodiments of the present disclosure.

In some embodiments, if the needle insertion assembly 12 includes two needle insertion teeth, the two needle insertion teeth 123 are assembled at two ends of the slider body 1241, as shown in FIG. 2 and FIG. 5A. The bottom ends of the two needle insertion teeth 123 snap together or separate from the suture needle 13, and a needle insertion tooth shrapnel 128 is provided between the top ends of the two needle insertion teeth 123 and the bottom end of the upper housing 125. The bottom end of the needle insertion tooth 123 may be an end of the needle insertion tooth 123 proximate to the suture needle, and the top end of the needle insertion tooth 123 may be an end of the needle insertion tooth 123 proximate to the upper housing.

In some embodiments, when the suture needle 13 squeezes the bottom ends of the two needle insertion teeth 123, the needle insertion tooth shrapnel 128 undergoes deformation, the two needle insertion teeth 123 contract upwardly, and the two needle insertion teeth 123 and the suture needle 13 separate. When the two needle insertion teeth 123 run into corresponding slots (e.g., a slot 132, etc.) on the suture needle 13, the needle insertion tooth shrapnel 128 returns and pushes the two needle insertion teeth 123 so that the bottoms of the two needle insertion teeth 123 snap into the corresponding slots on the suture needle 13. The present disclosure does not place any limitation on the retractable structure of the needle insertion tooth. In other embodiments, other materials that are capable of elastic deformation may be used in place of the needle insertion tooth shrapnel 128.

FIG. 4A is a schematic diagram illustrating a structure of a suture needle according to some embodiments of the present disclosure.

FIG. 4B is a schematic diagram illustrating a structure of the suture needle in another view according to some embodiments of the present disclosure.

FIG. 4C shows a side view of a suture needle according to some embodiments of the present disclosure.

In some embodiments, referring to FIG. 4A to FIG. 4C. A suture thread 131 may be fixed or removably fixed to one end of the suture needle 13, with the end of the suture needle 13 not connected to the suture thread 131 being a tip that pierces the target suturing tissue.

In some embodiments, the suture needle 13 has four slots 132 on one side and three slots 132 on the other side. By setting in this way, a 60° and/or 120° needle insertion of the suture needle can be realized, making the stroke control of the insertion of the suture needle 13 more flexible. If only a 180° needle insertion of the suture needle needs to be controlled, there is no need to set up a plurality of slots.

In some embodiments, in order to prevent the thickness of the suture needle from thinning, the slots on both sides may be set in a staggered manner, i.e., the slots are not provided on the corresponding two sides of the same position. With such a setting, the suture needle is not easily bent out of shape or broken. The present disclosure does not impose any limitation on the manner in which the slots are set up.

In some embodiments, a tooth portion of the needle insertion tooth 123 is detachably engaged in the slot 132 of the suture needle 13. The present disclosure does not limit a count of slots.

In some embodiments, each slot 132 is an asymmetric structure. One side of each slot 132 is a beveled surface with a larger inclination angle (e.g., a beveled surface a with a larger inclination angle, etc.), which acts as a barrier, while the other side is a beveled surface with a smaller inclination angle (e.g., a beveled surface b with a smaller inclination angle, etc.), which matches the top beveled surface of the tooth portion of the needle insertion tooth 123. The top of the tooth portion of the needle insertion tooth 123 is inclined. The top of the tooth portion may be an end of the tooth portion near the slot.

In some embodiments, the top beveled surface of the tooth portion of the needle insertion tooth 123 is able to slide out of the slot 132 through the beveled surface b under the force of the drive component 121, thereby releasing the engagement relationship between the needle insertion tooth 123 and the suture needle 13.

In some embodiments, a range of an angle between the beveled surface a of each slot 132 and one side surface c of the suture needle 13 may be pre-set (e.g., 60°-90° or 70°-80°, etc.), and a range of an angle between the beveled surface b and the side surface c of the suture needle 13 may be pre-set (e.g., as 10°-45° or 20°-35°, etc.). The present disclosure does not place any limitation on the specific degree of the inclination angle. In some embodiments, the beveled surface b is a non-planar surface such as a curved surface or the like, which is more conducive for the top of the tooth portion of the needle insertion tooth 123 to slide out of the slot 132, thereby disengaging the engagement relationship between the needle insertion tooth 123 and the slot 132.

In some embodiments, the suture needle 13 is a suture needle with thread. The tail portion of the suture needle 13 (the end to which the suture thread is attached) has a conical depression. One end of a suture thread 131 is tucked into the conical depression, and the suture thread 131 is detachably fixed in the conical depression of the suture needle 13 through squeezing or gluing, or the like. The present disclosure does not place any limitation on the manner in which the suture thread is threaded. In other embodiments, the suture thread may be removably fixed to one end of the suture needle in a plurality of ways, such as by conventional needle threading.

In some embodiments, the material of the suture needle 13 may include stainless steel, etc. The diameter of the cross-section of the suture needle 13 may be pre-set, such as 8 mm, 10 mm, 12 mm, etc. The present disclosure does not limit the size of the suture needle, and an appropriate size suture needle may be selected according to the actual therapeutic needs.

In some embodiments, the length range of the suture thread 131 may be pre-set, such as 30 cm-60 cm, 40 cm-50 cm, etc. The material of the suture thread 131 may include a polypropylene thread, a nylon thread, or the like. The present disclosure also does not place any limitation on the length, thread diameter, and material of the suture thread. In other embodiments, the suture thread 131 may include a bioabsorbable suture thread, realizing a subsequent operation without suture removal and reducing the risk of a secondary surgery for the patient.

In some embodiments, the anti-retraction component 14 is disposed on the second housing 112 and forms the separable engagement relationship with the suture needle 13, thereby preventing the suture needle 13 from retracting. The needle insertion assembly 12 and the anti-retraction component 14 are disposed on both sides of the suture needle 13, respectively.

In some embodiments, as shown in FIG. 2, the anti-retraction component 14 includes a lower housing 141, an anti-retraction tooth 142, and an anti-retraction tooth shrapnel 143. The lower housing 141 is capped at the bottom of the second housing 112, and the lower housing 141 has two positioning holes 1411. Two anti-retraction teeth 142 are mounted in the two positioning holes 1411, respectively, and the bottom ends of the two anti-retraction teeth 142 are connected to the anti-retraction tooth shrapnel 143. In actual assembly, the suture needle 13 is first positioned in the sliding groove 1121 of the second housing 112, after which the lower housing 141 equipped with the anti-retraction tooth 142 is capped to the bottom of the sliding groove 1121, and then the anti-retraction tooth shrapnel 143 is fixed to the bottom of the lower housing 141.

In some embodiments, the top of the anti-retraction tooth 142 may be seated on the slot 132 of the suture needle 13. In some embodiments, the tops of the two anti-retraction teeth 142 are provided with beveled surfaces that match the inclination angle of the beveled surfaces b of the slot 132 so that the anti-retraction teeth 142, under the force of the drive component 121, can slide out of the slot 132 through the beveled surface b. The top of the anti-retraction tooth 142 may be an end of the anti-retraction tooth 142 proximate to the suture needle. The bottom end of the anti-retraction tooth 142 may be an end of the anti-retraction tooth 142 away from the suture needle.

In some embodiments, the needle insertion tooth 123 and the anti-retraction tooth 142 are located on two sides of the suture needle 13, respectively, and independently detachably snap onto the slot 132 of the suture needle 13. Both the needle insertion tooth 123 and the anti-retraction tooth 142 are in the separable engagement relationship with the suture needle 13, so that complex functions (e.g., suturing of the target suturing tissue and knotting of the suture thread) can be achieved by simple structures, and the suturing device occupies a small volume and has a low production cost.

The present disclosure does not place any limitation on the shape of the tops of the needle insertion tooth 123 and the anti-retraction tooth 142. In other embodiments, the tops of the needle insertion tooth 123 and the anti-retraction tooth 142 may also be curved for easy sliding. As long as the interfacing structure that can form the engagement relationship with the suture needle and can be released from the engagement relationship is capable of realizing the function of the present disclosure, it is also within the scope of the present disclosure intended to be protected.

In some embodiments, the anti-retraction tooth shrapnel 143 relies on its elastic force to exert elastic pressure on the anti-retraction tooth 142, which can not only cause displacement of the anti-retraction tooth 142 when the suture needle 13 moves and slide out of the slot 132, but also cause the anti-retraction tooth 142 to snap into the slot 132 by the elastic pressure pressing the anti-retraction tooth 142.

In order to more clearly illustrate how the suturing device provided by the embodiments of the present disclosure performs a suturing action on the target suturing tissue, the following is illustrated in conjunction with FIG. 5A-FIG. 5F. In order to clearly show the location of the internal parts, not all of the components are drawn in the accompanying drawings. The “front side,” “back side,” “left,” and “right” referred to in the following descriptions are based on the positional relationships in FIG. 5A-FIG. 5F.

In some embodiments, the needle insertion tooth is located on the front side of the suture needle, and correspondingly, the anti-retraction tooth is located on the back side of the suture needle. The left side (a) of each of FIG. 5A-FIG. 5F shows a diagram of the connection relationship of the needle insertion assembly 12, the suture needle 13, and the anti-retraction component 14 when the drive component 121 is pulled, while the right side (b) shows a diagram of the connection relationship of the needle insertion assembly 12, the suture needle 13, and the anti-retraction component 14 when the drive component 121 is released and the return component 122 drives the needle insertion assembly 12 to return.

In some embodiments, from the end of the suture needle connected to the suture thread (the left end of the suture needle in the figure, also referred to as a tail end) to the tip of the suture needle (the right end of the suture needle in the figure), the four slots on the front side of the suture needle (the side close to the slider) are separately defined as a first front slot, a second front slot, a third front slot, and a fourth front slot. The three slots on the back side of the suture needle (the side away from the slider) are separately defined as a first back slot, a second back slot, and a third back slot.

The initial positions of the two needle insertion teeth 123, the suture needle 13, and the two anti-retraction teeth 142 in the suturing device in an initial state are shown in (b) in FIG. 5F. At the same time, this state is also the position of the suturing device after completing one suture, that is, the position to which the suturing device returns after completing one suture.

In some embodiments, the initial positions of the two needle insertion teeth 123, the suture needle 13, and the two anti-retraction teeth 142 in the suturing device at the beginning of the suturing are shown in (b) in FIG. 5F, and the two needle insertion teeth 123 of the needle insertion assembly 12 are seated on the first front slot and the third front slot of the suture needle 13, respectively. One of the anti-retraction teeth 142 of the anti-retraction component 14 (the one located on the right side, as shown in (b) in FIG. 5F, noted as a first anti-retraction tooth) snaps into the third back slot of the suture needle 13. At this time, the operator (e.g., a doctor, etc.) pulls the drive component 121 (e.g., a manipulating rope, etc.). Since the two needle insertion teeth 123 are respectively seated in the two slots 132 of the suture needle 13, and the direction of the force of the drive component 121 is to make the top of the needle insertion teeth 123 press against the side with the greater inclination angle of the slot 132, there is a linkage relationship between the needle insertion tooth 123 and the suture needle 13 at this time. Under the control of the control handle, the drive component 121 pulls the needle insertion teeth 123 so as to drive the entire suture needle 13 in a clockwise direction while the return component 122 is compressed. When the return component 122 is compressed to its limit, the slider 124 can no longer drive the needle insertion tooth 123 forward (i.e., the manipulating rope can no longer be pulled at this time), and at this time, the first anti-retraction tooth just snaps into the second back slot, as shown in (a) in FIG. 5A.

For the first anti-retraction tooth, when the drive component is pulled for the first time in the position shown in (b) in FIG. 5F, the direction of operation of the first anti-retraction tooth relative to the suture needle 13 is counterclockwise (in reality, the anti-retraction tooth 142 is not moving but the suture needle 13 is moving clockwise), and the first anti-retraction tooth can slide out through the beveled surface of the third back slot, and then move to the second back slot in (a) in FIG. 5A. In (a) in FIG. 5A, the first anti-retraction tooth may not cause any obstruction to the travel of the suture needle 13. In this process, the tip of the suture needle 13 exits the second housing 112 and passes through the target suturing tissue.

After that, the operator releases the drive component 121, i.e., the force acting on the slider 124 by the drive component 121 in the clockwise direction disappears. At this time, the return component 122 provides the force acting in the counterclockwise direction to the slider 124 and the insertion teeth 123, and the two insertion teeth 123 may pass through the beveled surfaces of the first front slot and the third front slot, respectively, to slide out and back counterclockwise, wherein the backward displacement is the displacement of the return component 122 being compressed. After returning, the needle insertion tooth 123 (which may be noted as the second needle insertion tooth) located on the left side is suspended from the exterior of the tail end of the suture needle 13, while the needle insertion tooth 123 (which may be noted as the first needle insertion tooth) located on the right side is seated in the second front slot, as shown in (b) in FIG. 5A. During this process, the suture needle 13 is pressed against the side of the second back slot with a higher inclination angle by the top of the first anti-retraction tooth, the first anti-retraction tooth may not be disengaged from the second back slot, and therefore the suture needle 13 may not follow the two needle insertion teeth 123 and undergo a backward movement.

After that, the operator pulls the manipulating rope for a second time, the first needle insertion tooth snaps into the second front slot, and the direction of the force exerted by the manipulating rope is to make the top of the first needle insertion tooth rest against the side of the second front slot with the greater inclination angle. At this time, there is an engagement relationship between the first needle insertion tooth and the suture needle 13. The suture needle 13 continues to move clockwise along with the first needle insertion tooth and gradually passes through the target suturing tissue until the return component 122 is compressed to its limit, as shown in (a) in FIG. 5B. At this point, when it is not possible to pull the manipulating rope any further, the first anti-retraction tooth just snaps into the first back slot. Next, the operator releases the drive component 121 again, the compressed return component 122 provides a counterclockwise force to the first anti-retraction tooth, the slider 124 and the two needle insertion teeth 123 move backward counterclockwise, and the first needle insertion tooth snaps into the first front slot, as shown in (b) of FIG. 5B. The first anti-retraction tooth snaps into the first back slot, and the suture needle 13 does not retract.

After that, the operator pulls the manipulating rope for a third time, and the first needle insertion tooth links the suture needle 13. The suture needle 13 continues to run clockwise and passes through the target suturing tissue. When the manipulating rope is pulled to its limit for the third time, as shown in (a) of FIG. 5C, the anti-retraction tooth 142 (noted as the second anti-retraction tooth) located on the left side is engaged in the third back slot of the suture needle. After releasing the drive component 121 for the third time, as shown in (b) of FIG. 5C, the slider 124 and the two needle insertion teeth 123 move counterclockwise, the second needle insertion tooth snaps into the fourth front slot, the second anti-retraction tooth snaps into the third back slot, and the suture needle 13 does not retract.

Next, the operator pulls the manipulating rope for a fourth time, the second needle insertion tooth seated in the fourth front slot continues to drive the suture needle 13 clockwise to the compression limit of the return component 122, as shown in (a) of FIG. 5D. At this point, the tip of the suture needle 13 passes through the target suturing tissue and is back-needled into the second housing 1121 of the second housing 112, and the second anti-retraction tooth snaps into the second back slot of the suture needle. After releasing the drive component 121 for the fourth time, as shown in (b) of FIG. 5D, the slider 124 and the needle insertion tooth 123 are backed up counterclockwise, the second needle insertion tooth is seated in the third front slot, the second anti-retraction tooth is seated in the second back slot, and the suture needle 13 does not back up.

Continuing to pull the manipulating rope for a fifth time, the second needle insertion tooth seated in the third front slot drives the suture needle 13 to continue rotating clockwise into the sliding groove 1121 until it reaches the compression limit of the return component 122, as shown in (a) of FIG. 5E. At this point, the second anti-retraction tooth snaps into the first back slot of the suture needle. After releasing the drive component 121 for the fifth time, as shown in (b) of FIG. 5E, the slider 124 and the two needle insertion teeth 123 are backed up counterclockwise, the two needle insertion teeth 123 are seated in the second front slot and the fourth front slot, respectively, the second anti-retraction tooth is seated in the first back slot, and the suture needle 13 does not back up.

Finally, the drive component 121 is pulled for a sixth time to the compression limit of the return component 122. The two needle insertion teeth 123, which are seated in the second front slot and the fourth front slot, continue to drive the suture needle 13 forward until the tip of the suture needle approaches the outlet end of the sliding groove 1121, as shown in (a) of FIG. 5F. At this point, the suture needle 13 drives the suture thread 131 to pass through the target suturing tissue, completing a suture. The first anti-retraction tooth snaps into the third back slot. After that, the drive component 121 is released for the sixth time, and as shown in (b) of FIG. 5F, the return component 122 drives the slider 124 and the two needle insertion teeth 123 back counterclockwise, the two needle insertion teeth 123 snap into the first front slot and the third front slot, respectively. The suture needle 13 is fully back into the second housing in preparation for the next suture.

In some embodiments, the compression displacement of the return component 122 (i.e., the displacement through which the return occurs) is equal to the circumferential distance (which may be referred to as the slot spacing) between two adjacent slots 132 on the suture needle, while the circumferential distance between the two needle insertion teeth 123 is twice the slot spacing. This setting enables at least one needle insertion tooth to be seated in one of the front slots of the suture needle after each return, so that the engagement relationship between the needle insertion tooth and the suture needle can be formed immediately when the manipulating rope is pulled at a later time to immediately drive the suture needle 13 forward. The present disclosure does not limit this in any way.

In other embodiments, after resetting, the needle insertion tooth may also be disposed between the two front slots, at which point the needle insertion tooth is compressed upwardly by the suture needle. When pulling the manipulating rope next time, the compressed needle insertion tooth first moves along the surface of the suture needle for a distance until at least one needle insertion tooth is engaged in one of the front slots in the engagement relationship. For the two anti-retraction teeth, it is only necessary to satisfy that at any time after pulling the drive component, one of the anti-retraction teeth can be engaged in one of the back slots to prevent the suture needle from retracting.

The above six operations achieve one-time suture of the suture needle, and the above operation process is repeated to perform suturing of the target suturing tissue or knotting of the suture thread. The present disclosure does not place any limitation on a count of drawstring pulls required for one suture. In other embodiments, the count of drawstring pulls required for one suture can be adjusted by adjusting the compression displacement of the return component and the slot spacing on the suture needle.

In some embodiments, the operations of the drive component 121 may be performed by the control handle for tensioning or releasing. The present disclosure does not limit this in any way. In other embodiments, the drive component 121 may also be other components that can drive the movement of the slider.

In some embodiments, the present disclosure further provides a therapeutic apparatus, the therapeutic apparatus includes the suturing device and the control handle as referred to above, and the therapeutic apparatus is used in conjunction with the endoscope. The suturing device is fixed to the apex of the endoscope. The control handle is fixed to the operating end of the endoscope, the drive component of the suturing device is connected to the control handle, and the control handle controls the drive component to control the insertion of the suture needle.

FIG. 6A shows a schematic diagram of a structure of a single cord (rope) control handle according to some embodiments of the present disclosure.

FIG. 6B to FIG. 6D are schematic diagrams illustrating a structure of a single cord (rope) control handle in other views according to some embodiments of the present disclosure.

FIG. 6E is a disassembled schematic diagram of a single cord (rope) control handle according to some embodiments of the present disclosure.

FIG. 7 is a schematic diagram illustrating the assembly of a manipulating rope pulley and a handle front housing on a single cord (rope) control handle according to some embodiments of the present disclosure.

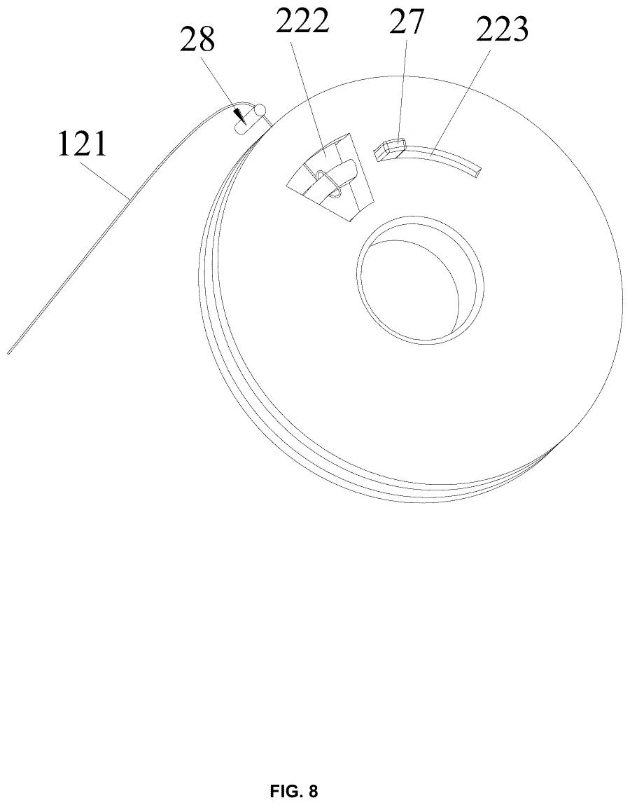

FIG. 8 is a schematic diagram illustrating a structure of a manipulating rope connected to the manipulating rope pulley using a circular pin and a pin spring according to some embodiments of the present disclosure.

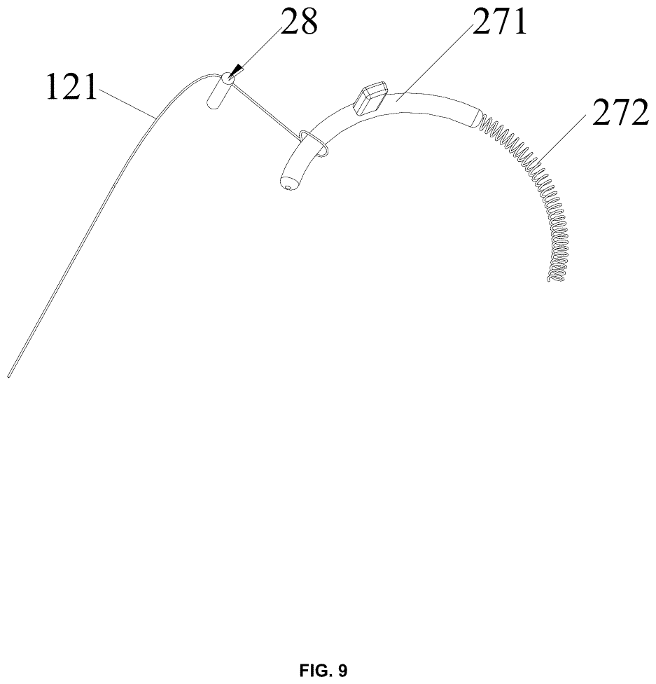

FIG. 9 is a schematic diagram illustrating the assembly of a manipulating rope, a circular pin, and a pin spring according to some embodiments of the present disclosure.

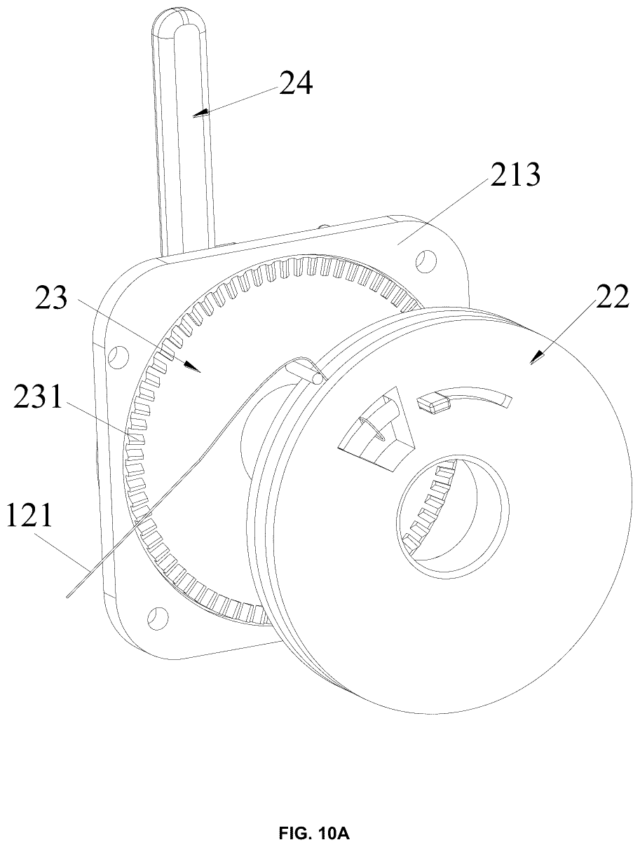

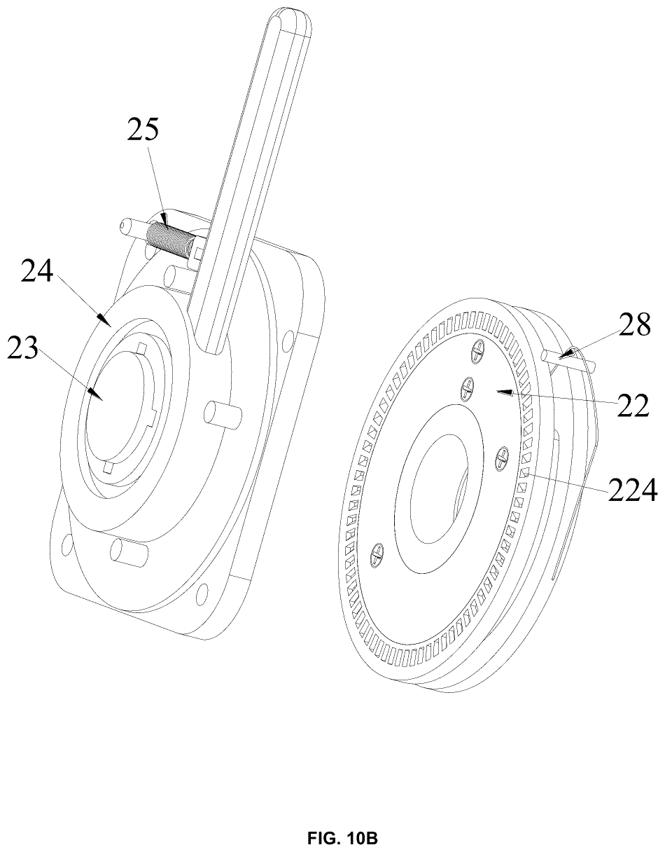

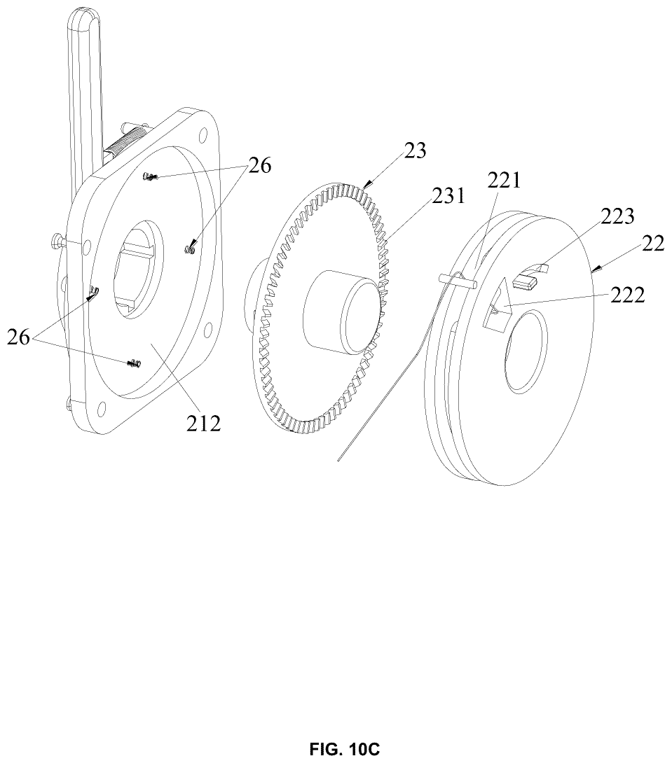

FIG. 10A is a schematic diagram illustrating the assembly of a pull rod, a handle rear housing, a clutch disk, and a manipulating rope pulley of a control handle according to some embodiments of the present disclosure.

FIG. 10B is a schematic diagram illustrating a structure of a pull rod, a handle rear housing, a clutch disk, and a manipulating rope pulley of a control handle in another view according to some embodiments of the present disclosure.

FIG. 10C is a disassembled schematic diagram of a pull rod, a handle rear housing, a clutch disk, and a manipulating rope pulley of a control handle according to some embodiments of the present disclosure.

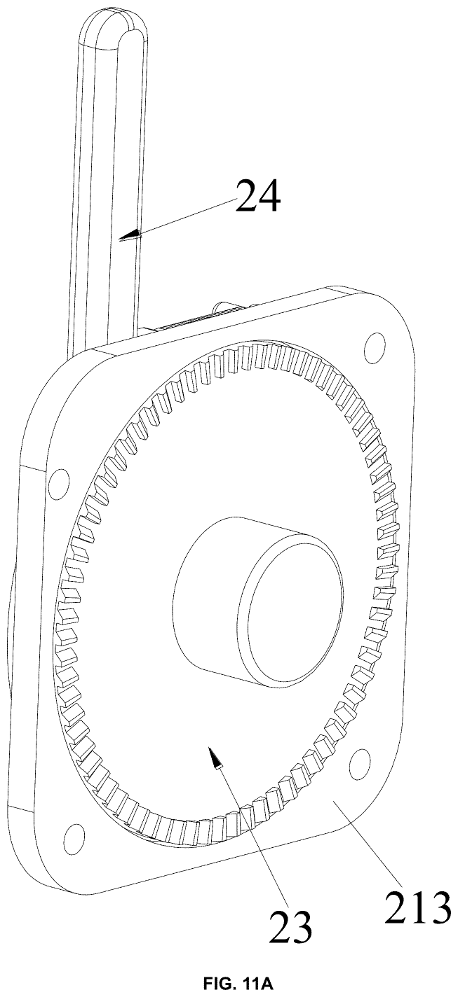

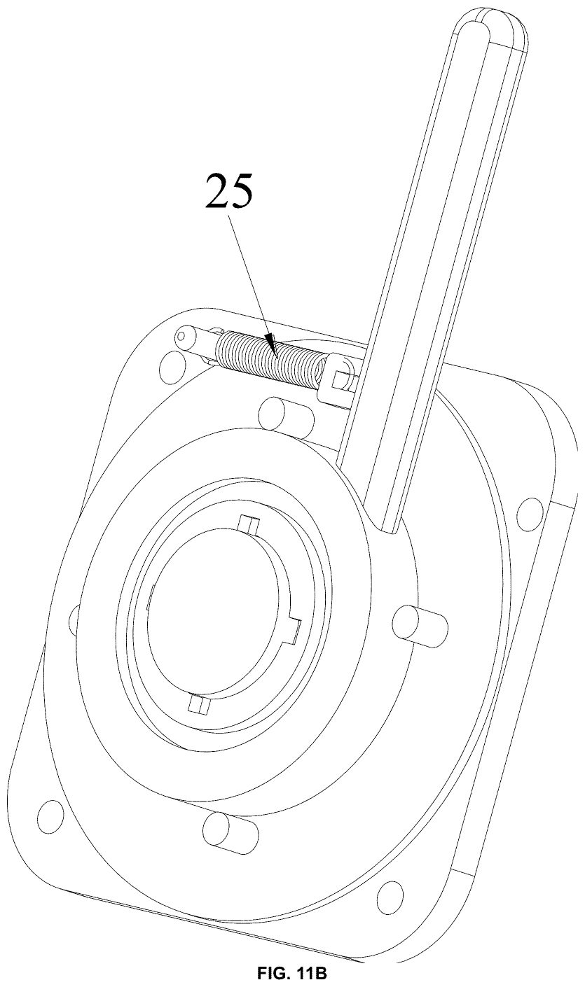

FIG. 11A is a schematic diagram illustrating the assembly of a pull rod, a handle front housing, and a clutch disk of a control handle according to some embodiments of the present disclosure.

FIG. 11B is a schematic diagram illustrating a structure of a pull rod, a handle front housing, and a clutch disk of a control handle in another view according to some embodiments of the present disclosure.

In some embodiments, as shown in FIG. 6A to FIG. 11B, a control handle 2 includes a handle housing 21, a manipulating rope pulley 22, a clutch disk 23, and a pull rod 24. The manipulating rope pulley 22 is provided in the handle housing 21, and the drive component 121 on the suturing device is the manipulating rope and is connected to the manipulating rope pulley 22. The clutch disk 23 is detachably disposed on one side of the manipulating rope pulley 22 (e.g., on the side of the manipulating rope pulley 22 relative to the interior of the handle housing 21), and the clutch disk 23 is provided with a snap ring 231 on the side proximate to the manipulating rope pulley 22 that engages with the manipulating rope pulley 22. The pull rod 24 is attached to the clutch disk 23 and is resettably attached to the handle housing 21.

In some embodiments, as shown in FIG. 11A and FIG. 11B, the pull rod 24 is resettably connected to the handle housing 21 via a tension spring 25. The present disclosure does not limit in any way the manner in which the pull rod is connected to the handle housing. In other embodiments, the pull rod may also be provided with a torsion spring to connect to the handle housing to achieve reset.

In some embodiments, there is a detachable structure between the manipulating rope pulley 22 and the clutch disk 23 in the control handle 2. When it is necessary to insert the suture needle 13 of the suturing device 1 (not shown in the figure), the clutch disk 23 is connected to the manipulating rope pulley 22, and the clutch disk 23 is in a linked relationship with the manipulating rope pulley 22, thereby tensioning the manipulating rope. The operator pulls the pull rod 24, which rotates the manipulating rope pulley 22 through the clutch disk 23, thereby enabling the pulling of the manipulating rope on the suturing device 1. After the manipulating rope is pulled to a limit position of the return component 122, the manipulating rope cannot be pulled anymore. At this time, the clutch disk 23 and the manipulating rope pulley 22 are separated, and the manipulating rope is in a relaxed state. The relaxed manipulating rope under the action of the return component 122 on the suturing device follows the slider 124 to pull back. The separated clutch disk 23 is driven by the tension spring 25 to return to its original position along with the pull rod 24, and the clutch disk 23 returns to its linked relationship with the manipulating rope pulley 22 after returning to its original position.

In some embodiments, as shown in FIG. 6E, the handle housing 21 includes a front housing 211, a middle housing 212, and a rear housing 213 arranged in sequence. The pull rod 24 is connected to the rear housing 213 via the tension spring 25. A rear shaft of the clutch disk 23 passes through the middle housing 212 and is provided on the pull rod 24. There are four compression springs 26 between the clutch disk 23 and the middle housing 212. The clutch disk 23 is engaged in the manipulating rope pulley 22 on the side near the manipulating rope pulley 22, and a front shaft of the clutch disk 23 passes through the manipulating rope pulley 22 and the front housing 211, respectively. When the drive component 121 is in a driving state, the four compression springs 26 push the clutch disk 23 to make the clutch disk 23 engage with the manipulating rope pulley 22. When the drive component can no longer be pulled, the four compression springs 26 are compressed by pressing the front shaft of the clutch disk 23 of the front housing 211. The clutch disk 23 is detached from the manipulating rope pulley 22 in the axial direction, and the manipulating rope is in a relaxed state. The return component 122 on the suturing device pulls back the relaxed drive component 121 via the slider 124. After releasing the pull rod 24, the pull rod 24 drives the clutch disk 23 to return under the action of the tension spring 25. The present disclosure does not place any limitation on a count of compression springs, and also does not place any limitation on the separation manner of the clutch disk.

In some embodiments of the present disclosure, the setting of the clutch disk 23 not only realizes the rotation of the manipulating rope pulley 22 following the pull rod 24, but also restricts the manipulating rope pulley 22 to avoid the manipulating rope pulley 22 rotating counterclockwise. The clutch disk 23 and the manipulating rope pulley 22 are engaged through gear matching, fully utilizing the principles of mechanics, increasing the mechanical torque and making the pull rod 24 more effortless.

In some embodiments, the manipulating rope pulley 22 has a slot ring 224 corresponding to the snap ring 231, and when engaged, the teeth on the snap ring 231 are inserted into the slot on the slot ring 224. The present disclosure does not limit the setting of the slot ring in any way. In other embodiments, the manipulating rope pulley may also be provided with protruding teeth that can engage with the snap ring.

In some embodiments, as shown in FIG. 7 to FIG. 9, the control handle 2 further includes a manipulating rope retainer 27 disposed within the manipulating rope pulley 22, and the manipulating rope serving as the drive component 121 is connected to the manipulating rope pulley 22 via the manipulating rope retainer 27. The manipulating rope retainer 27 includes a circular pin 271 and a pin spring 272, and the circular pin 271 is connected to the inside of the manipulating rope pulley 22 via the pin spring 272.

In some embodiments, the manipulating rope pulley 22 is provided with a side hole 221 and a latch sliding groove 223. The drive component 121 extends into the manipulating rope pulley 22 through the side hole 221 and is fitted with the circular pin 271, which ensures that the drive component 121 will not be detached from the manipulating rope pulley 22 during the pulling process. When fixing the drive component 121, an operator first pulls the circular pin 271 clockwise along the latch sliding groove 223, and then extends the drive component 121 through the side hole 221 of the manipulating rope pulley 22 into the manipulating rope pulley 22. A retaining ring at the front end of the drive component 121 is aligned with an end of the circular pin 271. The operator releases the circular pin 271, the pin spring 272 drives the circular pin 271 to return along the latch sliding groove 223, and the front end of the circular pin 271 passes through the retaining ring at the front end of the manipulating rope pulley 22.

In order to conveniently view the fixation state of the drive component 121, in some embodiments, the manipulating rope pulley 22 also has a viewing hole 222. The setting of the manipulating rope retainer 27 makes the drive component 121 change along a direction of the circular arc wheel surface of the manipulating rope pulley within the control handle 2, greatly reducing the resistance of the drive component 121 to run. It is very easy to pull back the pull rod with fingers, and the operation is very convenient. The present disclosure does not limit the setting of the manipulating rope retainer in any way.

In some embodiments, the control handle 2 further includes a guide cylindrical pin 28 disposed on the wheel surface of the manipulating rope pulley 22 and for guiding the drive component 121. The drive component 121 bypasses the guide cylindrical pin 28 and enters the side hole 221. The present disclosure does not limit the setting of the guide cylindrical pin in any way.

After installation, due to the need to match different models of endoscopes, the length of the drive component 121 reserved outside the manipulating rope pulley 22 may vary, and there may be an excessive amount of reservation, which may cause the drive component 121 to be in a relaxed state. At this point, the clutch disk 23 is pressed to disengage the clutch disk 23 from the manipulating rope pulley 22, and then the manipulating rope pulley 22 is rotated counterclockwise to tighten the excess drive component 121 within the manipulating rope pulley 22. After tightening, the clutch disk 23 is released and the clutch disk 23 engages the manipulating rope pulley 22.

In some embodiments, the front housing 211, the middle housing 212, and the rear housing 213 are square structures. Compared to a circular housing, a square housing structure allows for a smaller size of the individual component inside, so the overall profile may be smaller, and thus the package may be smaller, greatly reducing costs.

In some embodiments of the present disclosure, the square housing also makes the distance between the pull rod 24 and the base of the thumb of the operator narrow, reduces the range of finger spreading and hooking, reduces the operating resistance, which is in line with the ergonomics of the hand and is not likely to induce damage to the operator's hand tendon sheath, ligament, or muscle, making the operation more relaxing, suturing more accurate, surgery more rapid, and the connection of the suture more lasting, and the finger fatigue slower. The present disclosure does not limit the structure of the handle housing in any way.

In some embodiments, the present disclosure further provides a therapeutic device, and the therapeutic device includes the therapeutic apparatus and the endoscope mentioned above. The therapeutic apparatus is used in conjunction with the endoscope, and the therapeutic apparatus includes the suturing device 1 and the control handle 2 mentioned above.

FIG. 12 is a schematic diagram illustrating the installation of a connecting ring with an endoscope and a push catheter according to some embodiments of the present disclosure.

In some embodiments, as shown in FIG. 12, the therapeutic device includes an endoscope 200, and the endoscope 200 includes an apex 202 of the endoscope and an operating end 201 of the endoscope. The therapeutic apparatus further includes an external clamp catheter 3, a connecting ring 4, a push catheter 5, a loop ring, and a hook clamp. The connecting ring 4 is socketed to the endoscope 200. The connecting ring 4 has a variable diameter, and may be constituted by at least one or a combination of self-locking clasp teeth or/and chains or/and pin pins, etc., to adapt to be mounted on the apex of the endoscope of different diameters, and the present disclosure does not make any limitation on the way the connecting ring is constituted. The loop ring performs a cutting action of the tissue at the apex of the endoscope 200, the hook clamp performs a clamping action of the tissue or a hooking and pulling action of the suture thread at the apex of the endoscope 200, and the push catheter 5 is configured to push forward longitudinally and assist in tightening the knot horizontally.

In some embodiments, the external clamp catheter 3 is fixed to the hose portion of the endoscope 200 by the connecting ring 4 located higher among the two connecting rings 4, and the push catheter 5 is disposed in the inner bore of the external clamp catheter 3. The connecting ring 4 located lower among the two connecting rings 4 is snapped onto the apex 202 of the endoscope 200, and the suturing device 1 is snapped onto the connecting ring 4, thereby securing the suturing device 1 to the apex 202 of the endoscope 200. The head end of the external clamp catheter 3 is inserted into the suturing device 1.

In some embodiments of the present disclosure, the connecting ring 4 can not only fix the external clamp catheter 3 and the endoscope 200, but also increase the connection strength of the apex 202 of the endoscope 200, so that the suturing device 1 can be firmly fixed to the apex 202 of the endoscope 200. In some embodiments, the connecting ring 4 is made of an elastic material such as silicone. The present disclosure does not limit the material of the connecting ring.

In some embodiments, the control handle 2 is mounted to the operating portion of the operating end 201 of the endoscope, and the control handle 2 and the endoscope 200 are securely fastened together by a handle fastener. If the handle fastener is a strap, the control handle 2 and the endoscope 200 are fastened by wrapping the strap around the endoscope 200. The present disclosure does not limit the types of handle fasteners in any way. Any component that can achieve a fixed function can be used as a handle fastener.

In some embodiments, the process of using the therapeutic device includes the following operations.

The operator turns the endoscope on, and under observation through the externally attached monitor, adjusts the orientation of the suturing device 1 to make the C-shaped opening face downward, and adjusts the suturing device to the target suturing tissue. After adjusting the orientation and position of the suturing device, the operator locks the suturing device until the stepped surface of the suturing device is tight against the apex of the endoscope.

The operator inserts the hook clamp from the insertion jaw of the endoscope, passes through the clamp channel to reach the apex 202 of the endoscope, and then clamps the drive component 121 of the suturing device, pulling the drive component 121 located near the apex 202 into the clamp channel. Then the operator takes out the hook clamp and pulls it out from the insertion jaw, thereby bringing one end of the drive component 121 out of the insertion jaw. Afterwards, the operator inserts the drive component 121 into the side hole 221 of the manipulating rope pulley 22 on the control handle 2, thereby fixing the drive component to the manipulating rope retainer 27.

The operator uses the loop ring to cut the target suturing tissue at the apex of the endoscope, or cuts the target suturing tissue through an electric current. Afterwards, the operator uses the suturing device to suture the target suturing tissue. The loop ring may be entered via the clamp catheter of the endoscope or through the external clamp catheter of the endoscope, without any limitation herein.

The operator utilizes the hook clamp to perform a clamping action of the target suturing tissue or a hooking and pulling action of the suture thread at the apex of the endoscope, and finally, the ends of the suture thread may be pinched to complete the knotting action of the suture thread. The operator utilizes the push catheter to push forward longitudinally and assist in tightening the knot horizontally at the apex of the endoscope. The hook clamp and the push catheter may be inserted through the clamp catheter of the endoscope, or they may be inserted through the external clamp catheter of the endoscope, and is not limited in any way here.

In practice, an operator may simultaneously insert two hook clamps or push catheters through two clamp channels for work, or use only one hook clamp or push catheter for work. In some embodiments, the front end of the hook clamp is in the shape of a clamp with a hook, and the size and material of the push catheter may be pre-set. For example, the length of the push catheter ranges from 500-2500 mm, the inner diameter ranges from 0.5 to 3.5 mm, the wall thickness ranges from 0.05-2.50 mm, and the material of the push catheter includes polymer materials such as medical polyamide or polytetrafluoroethylene. The present disclosure does not limit the size and material of the push catheter in any way.

In some embodiments of the present disclosure, through the loop ring, the suturing device, the hook clamp, and the push catheter, the therapeutic device and the therapeutic apparatus can achieve various functions such as cutting, suturing, and knotting of the target suturing tissue. In addition, the therapeutic device and the therapeutic apparatus may include a biological glue, a sealing glue, a stent, an anastomotic nail, and an anastomotic clip, thereby achieving diversified functions.

In another embodiment, the endoscope may be a dual clamp channel endoscope. At this point, there is no need to specifically install an external clamp catheter. Because the endoscope has two clamp channels, it is only necessary to insert the push catheter into the vacant clamp catheter.

The next step describes how the suturing device in the present disclosure performs suturing.

First, the operator inserts the hook clamp from the insertion jaw of the endoscope 200, passes through the clamp channel to reach the apex 202 of the endoscope, and locates and clamps the suture thread 131. The operator inserts the other hook clamp into the push catheter 5, reaches the apex 202 of the endoscope, clamps the edge of the target suturing tissue, and retracts smoothly to place the target suturing tissue in the middle of the C-shaped opening of the suturing device. Then the operator hooks the pull rod 24 to insert the suture needle 13, and when the needle is inserted to the limit position of the return component 122, the operator presses the front shaft of the clutch disk 23 on the control handle 2 to relax the drive component 121. The needle insertion assembly 12 retracts under the action of the return component 122. Afterwards, the operator repeats the above steps until the suture needle 13 rotates 360° within the sliding groove 1121 and returns to its initial position. The operator repeats the above steps to continuously suture.

After continuous suturing, the operator releases the hook clamp that holds the end of the suture thread 131. After the suture needle 13 has crossed over and been knotted halfway, the hook clamp is quickly moved to the front of the end of the suture thread 131 and the suture thread 131 is re-clamped. The operator tightens the two hook clamps while observing through the monitor, and gradually pushes the knot longitudinally with the push catheter 5 and assists in tightening the knot horizontally to achieve knotting. To ensure that the knot is tied securely enough, the operator may repeat the knotting operation again to complete the triple knot. The operator withdraws the push catheter 5 and the two hook clamps, inserts a scissor for the endoscope into the endoscopic vacant clamp catheter or the external clamp catheter, and snips the suture thread. After the suture is fully completed, the operator withdraws the endoscope and the ancillary tools, and removes the suturing device.

### Example 2

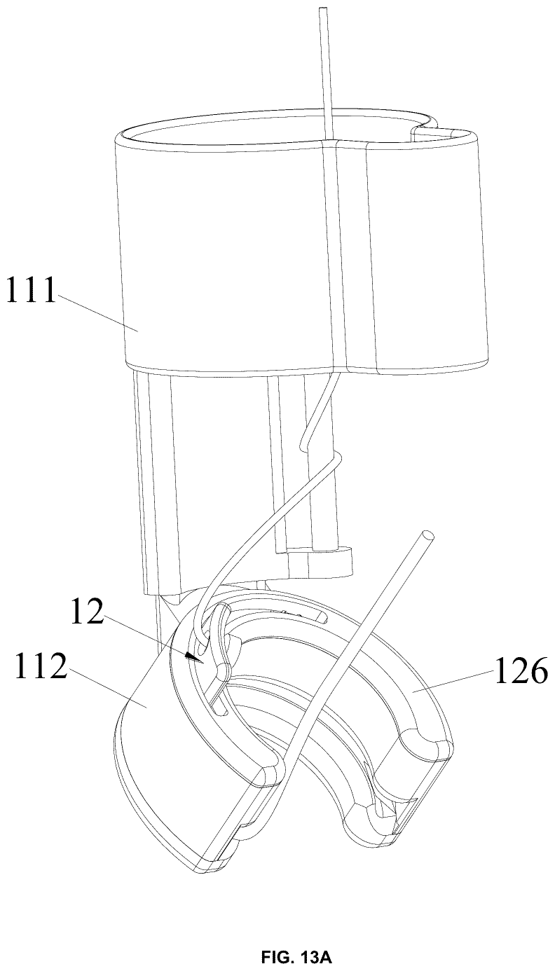

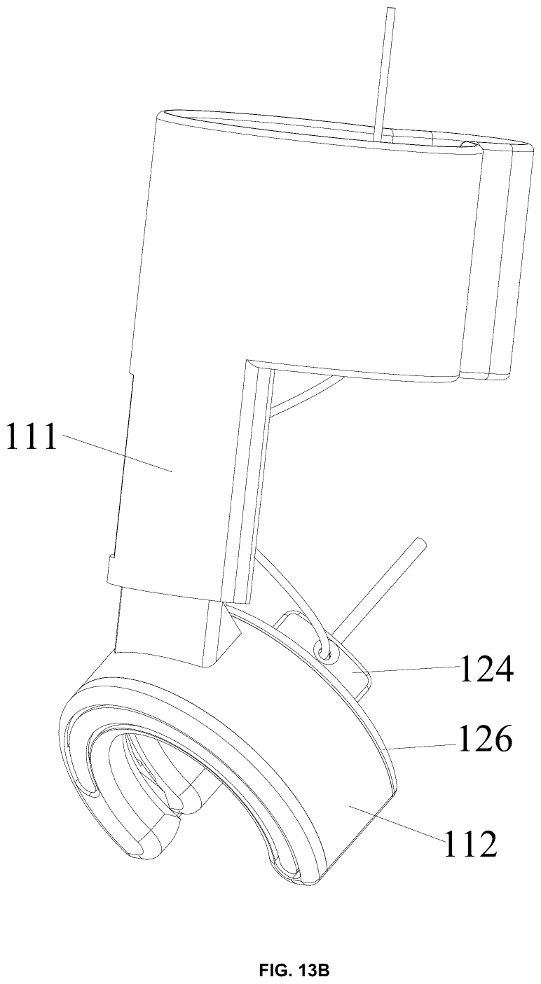

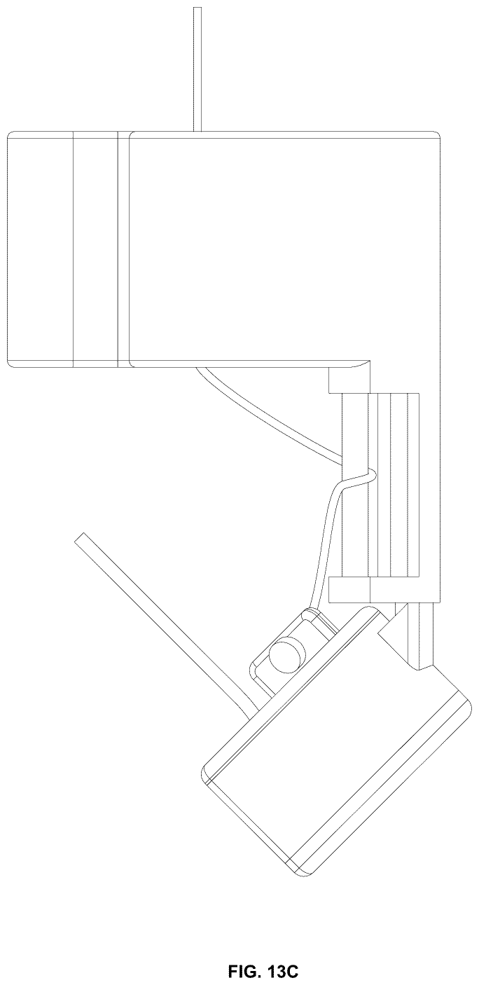

FIG. 13A is a schematic diagram illustrating a structure of a suturing device according to some other embodiments of the present disclosure.

FIG. 13B and FIG. 13C are schematic diagrams illustrating a structure of a suturing device in other views according to some other embodiments of the present disclosure.

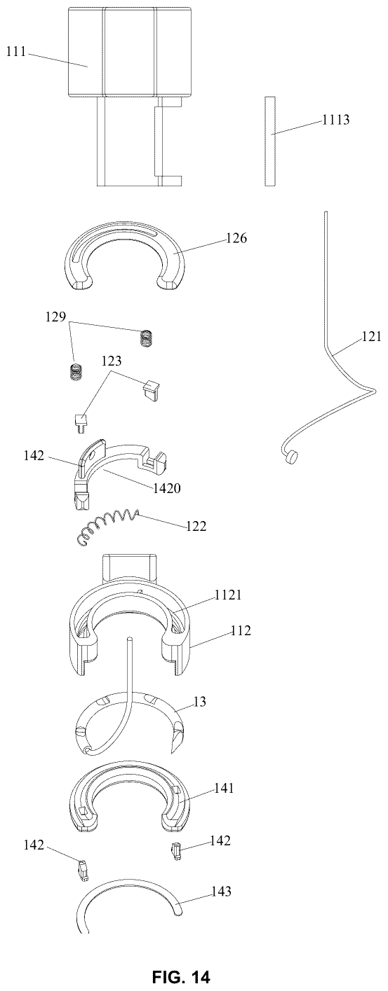

FIG. 14 shows a disassembled schematic diagram of a suturing device according to some other embodiments of the present disclosure.

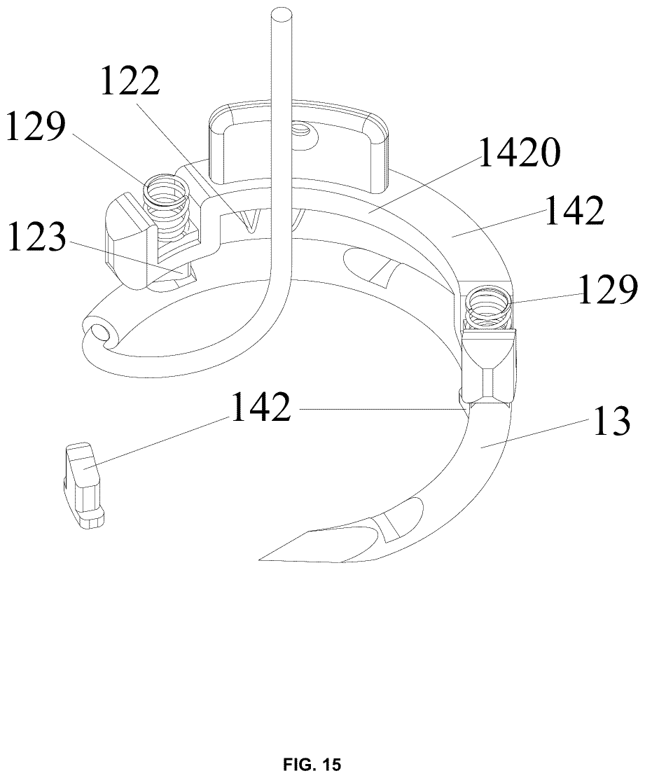

FIG. 15 is a schematic diagram illustrating the assembly of a suture needle and a needle insertion assembly in a suturing device according to some other embodiments of the present disclosure.

Example 2 is similar to Example 1, with the difference being that: the installation position of the return component 122 in the needle insertion assembly 12 is different, the upper housing is no longer provided in the needle insertion assembly 12, and the way of retracting and expanding of the needle insertion tooth 123 is different.

In some embodiments, referring to FIG. 13 to FIG. 15, the bottom of the slider 124 has a circular arc-shaped holding groove 1240, and the return component 122 is provided in the holding groove 1240 and is confined between the slider 124 and the suture needle 13. One end of the return component 122 is attached to a sidewall of the holding groove 1240, and the other end of the return component 122 is attached to an inner wall of the sliding groove 1121 of the second housing 112. The upper cover 126 is closed to the second housing 112. Similarly, the first housing 111 is connected to the second housing 112 through integrally molding.

In some embodiments, the return component 122 is disposed within the holding groove 1240, and the bottom wall of the slider 124 limits the direction of the deformation of the return component 122 to ensure that the return component 122 deforms in a circumferential direction of the sliding groove 1121.

Compared to the Example 1 that uses an upper housing to accommodate the return component, the setting of the holding groove 1240 in Example 2 eliminates the need to install the upper housing on the suturing device, greatly reduces the overall volume of the suturing device, not only realizing the miniaturization of the suturing device, but also greatly reducing the manufacturing cost.

In some embodiments, the needle insertion tooth 123 is attached to the upper cover 126 via the needle insertion tooth spring 129. One end of the needle insertion tooth spring 129 is connected to the upper cover 126, and the other end of the needle insertion tooth spring 129 is connected to the needle insertion tooth 123. When the needle insertion tooth 123 is seated in the slot of the suture needle 13, the needle insertion tooth spring 129 is in a naturally elongated state, whereas the needle insertion tooth spring 129 is compressed and the needle insertion tooth 123 is in a contracted state when the needle insertion tooth 123 is moved along the surface of the suture needle 13. The present disclosure does not make any limitations on the extension and retraction manner of the needle insertion tooth. In other embodiments, the extension and retraction of the needle insertion tooth may be achieved through the use of needle insertion tooth shrapnel.

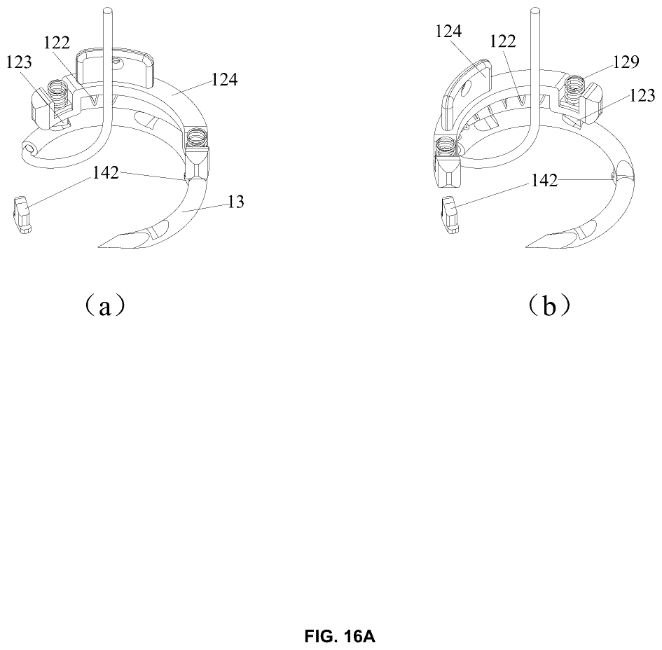

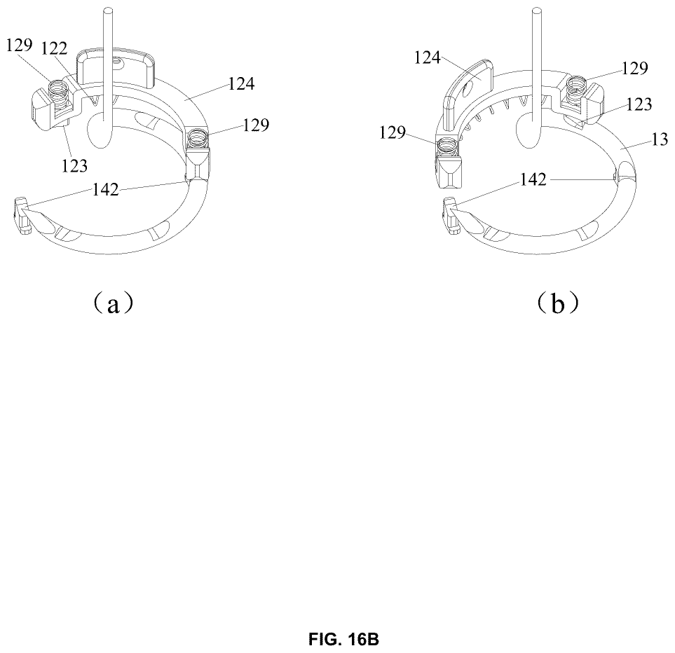

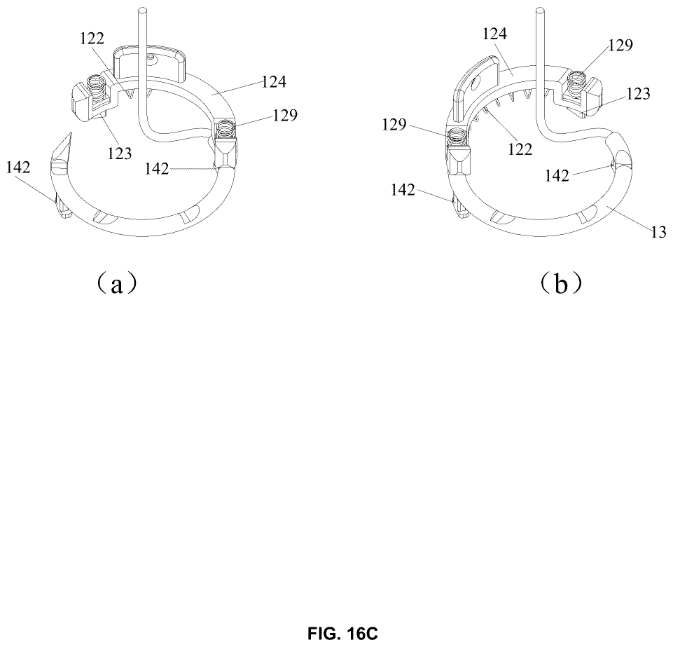

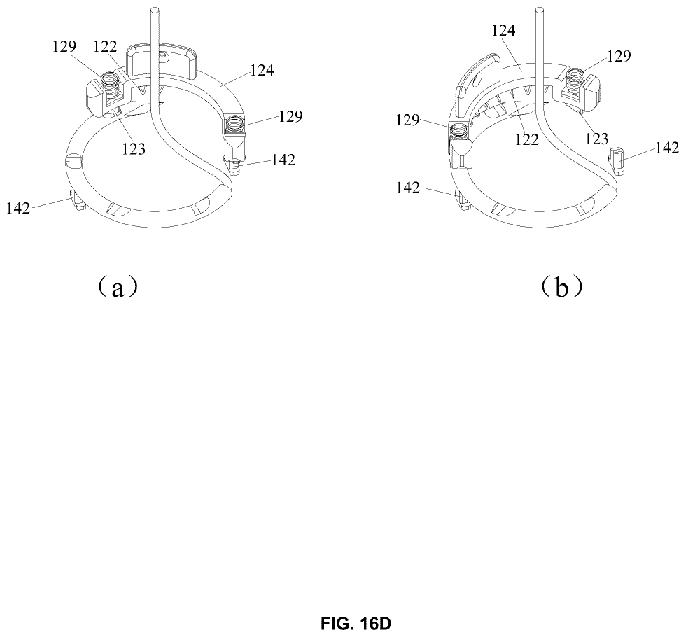

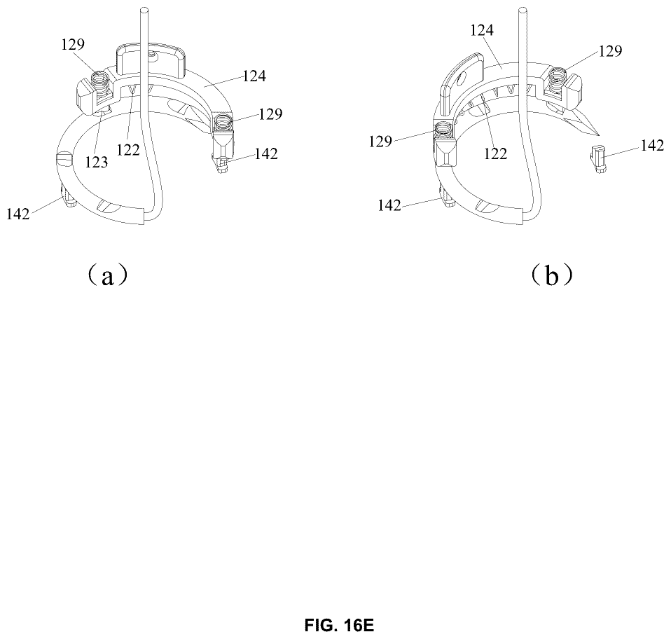

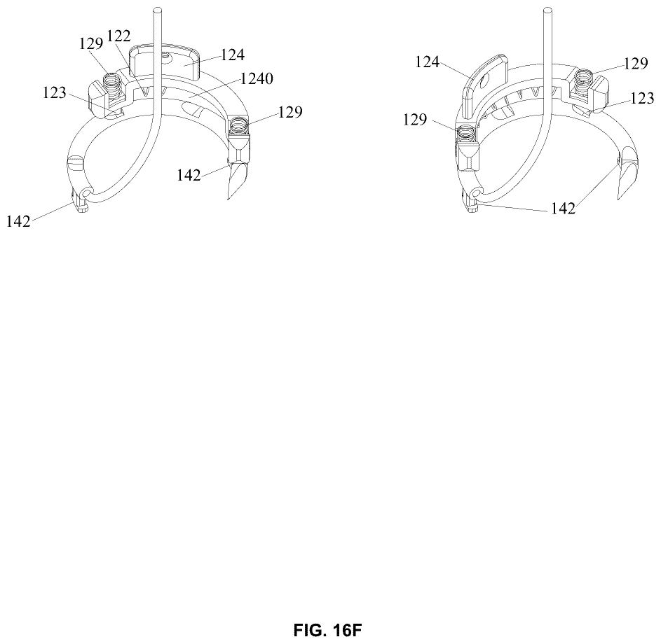

FIG. 16A to FIG. 16F show schematic diagrams of different states when an anti-retraction component, a suture needle, and a needle insertion assembly of a suturing device are in motion according to some other embodiments of the present disclosure. The motion state and suturing principle of the suturing device in Example 2 are the same as those in Example 1.

In some embodiments, as in Example 1, the anti-retraction component 14 of Example 2 further includes a lower housing 141, an anti-retraction tooth 142, and an anti-retraction tooth shrapnel 143. The first housing 111 also has an anti-tangle sliding column 1113.

### Example 3

FIG. 17A is a schematic diagram illustrating a structure of a suturing device according to some other embodiments of the present disclosure.

FIG. 17B and FIG. 17C are schematic diagrams illustrating a structure of a suturing device in other views according to some other embodiments of the present disclosure.

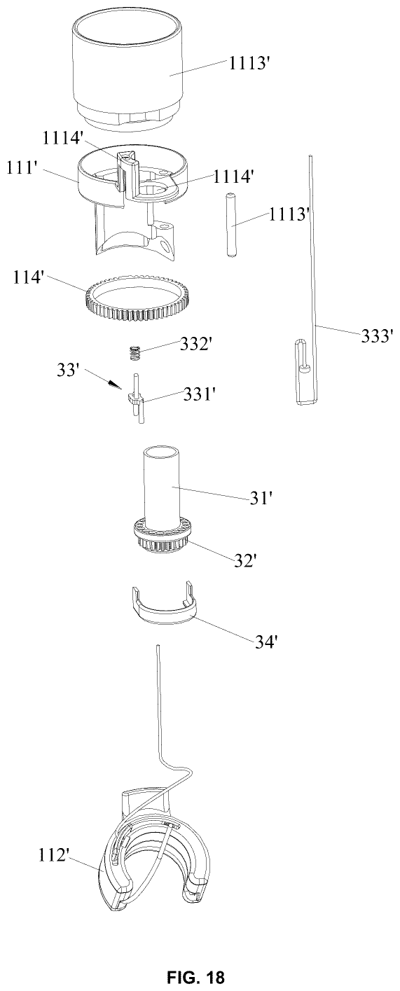

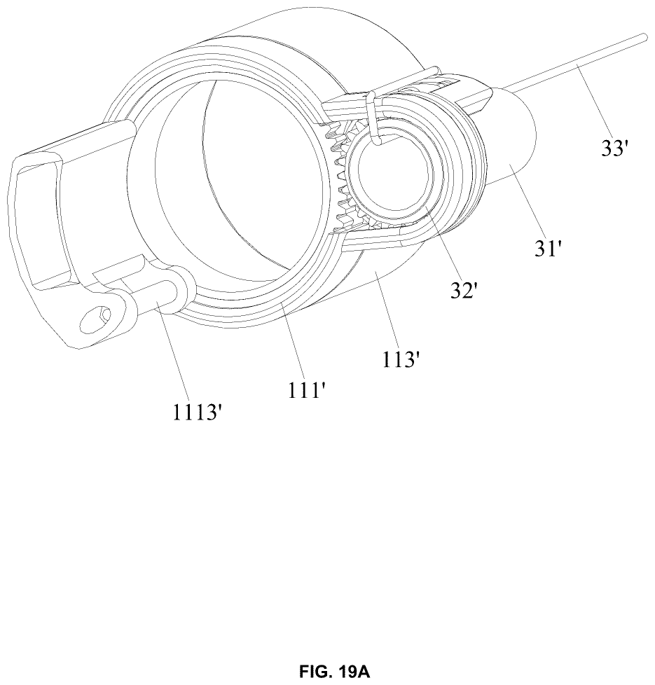

FIG. 18 shows a disassembled schematic diagram of a suturing device according to some other embodiments of the present disclosure.

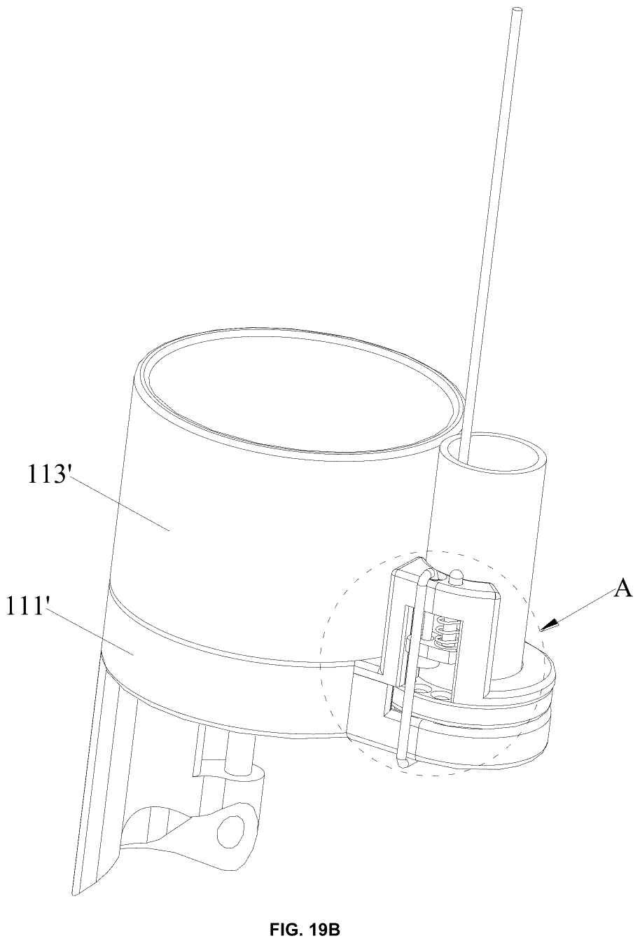

FIG. 19A is a schematic diagram illustrating the assembly of a second housing, a third housing, and an external clamp catheter of a suturing device according to some other embodiments of the present disclosure.

FIG. 19B is a schematic diagram illustrating a structure of a second housing, a third housing, and an external clamp catheter of the suturing device in another view according to some other embodiments of the present disclosure.

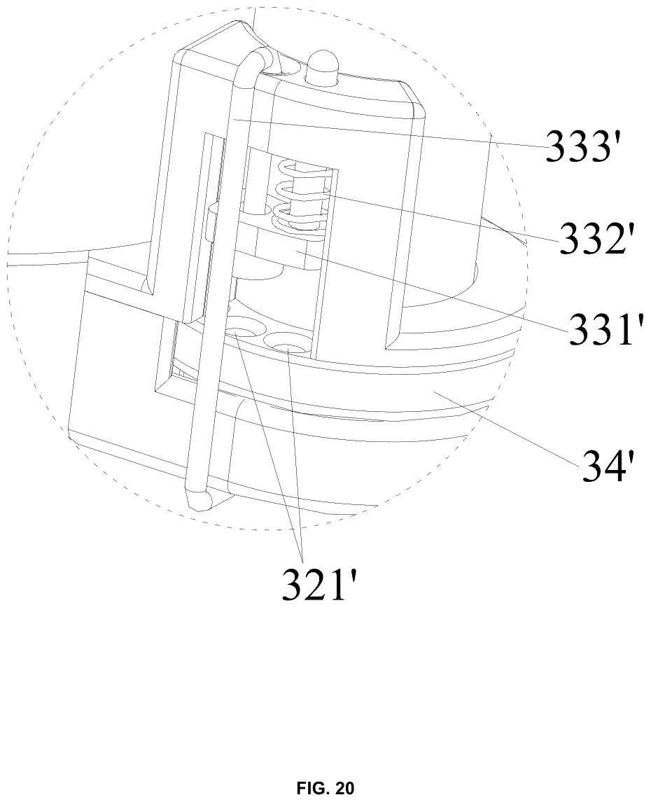

FIG. 20 shows a partially enlarged view of a second housing, a third housing, and an external clamp catheter of the suturing device according to some other embodiments of the present disclosure.

Example 3 is similar to Example 2, with the difference being that: the structure of the main housing 11′ is different.

In some embodiments, as shown in FIG. 17A to FIG. 20, the suturing device further includes an external clamp catheter 3′, and a main housing 11′ includes a first housing 111′, a second housing 112′, and a third housing 113′. The third housing 113′ and the second housing 112′ are disposed opposite to the first housing 111′, and the external clamp catheter 3′ is drivably connected to the first housing 111′ to drive the first housing 111′ and the second housing 112′ to rotate relative to the third housing 113′.

In some embodiments, the first housing 111′ and the second housing 112′ are integrally molded. The first housing 111′ is rotationally socketed to the front end of the third housing 113′, and the first housing 111′ has an anti-tangle sliding column 1113′. The present disclosure does not limit this in any way. In other embodiments, the first housing and the second housing may also be fixedly connected by connectors.

In some embodiments, as shown in FIG. 18, the main housing 11′ further includes a rotating gear 114′, and the first housing 111′ is rotationally connected to the third housing 113′ via the rotating gear 114′. The external clamp catheter 3′ includes a clamp catheter body 31′ and a clamp catheter drive tooth 32′. The clamp catheter body 31′ is assembled to a second channel on the first housing 111′. The clamp catheter drive tooth 32′ engages the rotating gear 114′. The external clamp catheter 3′ is driven by the clamp catheter drive tooth 32′ and the rotating gear 114′ to drive the first housing 111′ and the second housing 112′ to rotate. This setting greatly facilitates the adjustment of the orientation of the C-shaped opening of the suturing device in the patient's body, and it is very convenient to use, especially in the narrow body space.

The present disclosure does not limit the transmission structure between the first housing and the external clamp catheter, or the transmission between the first housing and the clamp catheter of the endoscope itself, and their respective relative movements. Other mechanical structures that can realize synchronous linkage to drive the second housing to rotate are within the scope of protection of the present disclosure.

In some embodiments, as shown in FIG. 18 and FIG. 20, the external clamp catheter 3′ further includes a locating component 33′, and the locating component 33′ locks the external clamp catheter 3′, so that the external clamp catheter 3′ cannot rotate or release the lock of the external clamp catheter 3′.

In some embodiments, FIG. 20 is an enlarged partial view of region A in FIG. 19B. As shown in FIG. 20, the locating component 33′ includes a locating pin 331′, a locating pin spring 332′, and a locating pin pull cope 333′. The clamp catheter drive tooth 32′ has a plurality of circumferential locating holes 321′, and one side of the second channel on the first housing 111′ has a locating component mounting portion 1114′. The upper end of the locating pin 331′ is fixed to the locating component mounting portion 1114′ by the locating pin spring 332′, and the lower end of the locating pin 331′ is opposed to a plurality of circumferential locating holes 321′ in the clamp catheter drive tooth 32′. The locating pin pull cope 333′ is attached to the locating pin 331′. The operator may cause the locating pin 331′ to extend or retract along the locating component mounting portion 1114′ by pulling the locating pin pull cope 333′, thereby causing the lower end of the locating pin 331′ to snap into or out of engagement with the plurality of circumferential locating holes 321′. When the locating pin 331′ snaps into the plurality of circumferential locating holes 321′ of the clamp catheter drive tooth 32′, the external clamp catheter 3′ is locked and cannot rotate, at which time the first housing 111′, the second housing 112′ and the third housing 113′ are fixed and do not rotate. When the operator pulls up the locating pin pull cord 333′ to disengage the lower end of the locating pin 331′ from the plurality of circumferential locating holes 321′, the clamp catheter drive tooth 32′ is unlocked, and the clamp catheter drive tooth 32′ can drive the first housing 111′ and the second housing 112′ to rotate relative to the third housing 113′.

The present disclosure does not limit the location and structure of the locating component. In other embodiments, the locating component may also be provided at the rotating gear 114′ to realize the transmission relationship between the first housing and the external clamp catheter by locking or unlocking the rotating gear.

In some embodiments, the locating pin pull cope 333′ may be wrapped downwards around the locating component mounting portion 1114′, and then passed through the clamp catheter body 31′ from bottom to top, making it convenient to pull the locating pin pull cope 333′. The locating pin pull cope 333′ may be fixed to the control handle or may be independently external to the control handle. The present disclosure does not limit this in any way.

In some embodiments, the clamp catheter drive tooth 32′ is also covered with a protective cover 34′. The present disclosure does not limit the setting of the protective cover in any way.

### Example 4

For larger wounds or other target suturing tissues, the operator uses the suturing device described in this present disclosure, as in the aforementioned embodiment, to insert and remove the needle from the target suturing tissue on one side of the wound, and then moves the C-shaped opening of the suturing device to the tissue on the opposite side of the larger wound or the target suturing tissue, and hooks the pull rod 24 to insert the suture needle 13. When the needle is inserted to the limit position of the return component 122, the operator presses the front shaft of the clutch disk 23 on the control handle 2 to relax the drive component 121, and the needle insertion assembly 12 retracts under the action of the return component 122. Afterwards, the operator repeats the above steps until the suture needle 13 rotates 360° within the sliding groove 1121 and returns to its initial position. The operator can repeat the above steps to continuously perform cross suturing on larger wounds or other target suturing tissues.

Although the present disclosure has been disclosed by the preferred embodiments as described above, it is not intended to limit the present disclosure. Any person skilled in the art may make some changes and modifications without departing from the spirit and scope of the present disclosure. Therefore, the scope of protection of the present disclosure shall be subject to the scope of protection required by the claims.

What is claimed is:

1. A suturing device, comprising:
a main housing;
a suture needle slidingly disposed within the main housing, wherein one end of the suture needle connects to a suture thread and the other end of the suture needle is a tip;
a needle insertion assembly disposed within the main housing, the needle insertion assembly including a drive component, a return component, and a needle insertion tooth, the needle insertion tooth being in a separable engagement relationship with the suture needle, wherein
when the drive component acts on the needle insertion tooth and the suture needle that are in an engagement state,
the suture needle is controlled to be inserted, and the return component undergoes deformation when the suture needle is inserted;
when the drive component is released, the engagement state of the needle insertion tooth and the suture needle is released, and the return component drives the needle insertion tooth to return; or
when the drive component acts on the needle insertion tooth that is in a released engagement state,
the needle insertion tooth moves relative to the suture needle, and the return component undergoes deformation when the needle insertion tooth moves relative to the suture needle;
when the drive component is released, the needle insertion tooth engages with the suture needle, and the return component drives the needle insertion tooth and the suture needle to insert; and
an anti-retraction component disposed within the main housing, the anti-retraction component forming a separable engagement relationship with the suture needle to prevent the suture needle from retracting.

2. The suturing device of claim 1, wherein the drive component includes a manipulating rope that acts on the needle insertion tooth, and the return component includes at least one of a return spring or a metallic spring plate that acts on the needle insertion tooth.

3. The suturing device of claim 1, wherein
the main housing has a sliding groove,
the suture needle is disposed in the sliding groove,
the needle insertion assembly further includes a slider disposed in the sliding groove and located above the suture needle,
the needle insertion tooth is retractably assembled in the slider, and
the drive component and the return component are both connected to the slider.

4. The suturing device of claim 3, wherein
the needle insertion assembly further includes an upper housing that covers the main housing and an upper cover that covers the upper housing,
a holding cavity is formed between the upper housing and the upper cover,
the slider includes a slider body and a slider connecting portion,
the slider connecting portion sequentially passes through the upper housing and the upper cover,
the return component is disposed within the holding cavity,
one end of the return component is connected to the slider connecting portion,
the other end of the return component is connected to the upper housing or the upper cover, and
the drive component is connected to an upper end of the slider connecting portion.

5. The suturing device of claim 3, wherein
a bottom of the slider has a holding groove,
the return component is disposed in the holding groove and is confined between the slider and the suture needle,
one end of the return component is connected to a side wall of the holding groove, and
the other end of the return component is connected to an inner wall of the sliding groove of the main housing.

6. The suturing device of claim 1, further comprising:
an external clamp catheter, wherein
the main housing includes a first housing, a second housing, and a third housing,
the first housing is connected to the second housing,
the third housing and the second housing are rotated relative to the first housing, and
the external clamp catheter is drivably connected to the first housing to drive relative rotation among the first housing, the second housing, and the third housing, so as to arbitrarily convert an orientation and a position of a C-shaped opening of the suturing device.

7. A therapeutic apparatus, the therapeutic apparatus being used in conjunction with an endoscope, wherein the therapeutic apparatus comprises:
a suturing device fixed at an apex of the endoscope, and the suturing device including:
a main housing;
a suture needle slidingly disposed within the main housing, wherein one end of the suture needle connects to a suture thread and the other end of the suture needle is a tip;
a needle insertion assembly disposed within the main housing, the needle insertion assembly including a drive component, a return component, and a needle insertion tooth, the needle insertion tooth being in a separable engagement relationship with the suture needle, wherein
when the drive component acts on the needle insertion tooth and the suture needle that are in an engagement state,
the suture needle is controlled to be inserted, and the return component undergoes deformation when the suture needle is inserted;
when the drive component is released, the engagement state of the needle insertion tooth and the suture needle is released, and the return component drives the needle insertion tooth to return; or
when the drive component acts on the needle insertion tooth that is in a released engagement state,
the needle insertion tooth moves relative to the suture needle, and the return component undergoes deformation when the needle insertion tooth moves relative to the suture needle;
when the drive component is released, the needle insertion tooth engages with the suture needle, and the return component drives the needle insertion tooth and the suture needle to insert; and
an anti-retraction component disposed within the main housing, the anti-retraction component forming a separable engagement relationship with the suture needle to prevent the suture needle from retracting; and
a control handle fixed at an operating end of the endoscope, wherein the drive component of the suturing device is connected to the control handle, and the control handle controls the drive component to control the insertion of the suture needle.

8. The therapeutic apparatus of claim 7, wherein the control handle includes:
a handle housing;
a manipulating rope pulley disposed within the handle housing, wherein the drive component of the suturing device is a manipulating rope, and the manipulating rope is connected to the manipulating rope pulley;
a clutch plate separability disposed on one side of the manipulating rope pulley, wherein the clutch plate has a snap ring engaged with the manipulating rope pulley on a side near the manipulating rope pulley; and
a pull rod connected to the clutch plate and resettably connected to the handle housing.

9. The therapeutic apparatus of claim 7, further comprising:
a connecting ring, a loop ring, a hook clamp, and a push catheter, wherein
the connecting ring is fitted onto the endoscope,
the loop ring performs a cutting action on a target suturing tissue at the apex of the endoscope, and
the hook clamp performs a grabbing action on the target suturing tissue or a hooking and pulling action on the suture thread at the apex of the endoscope.

10. A therapeutic device, comprising:
an endoscope;
a therapeutic apparatus used in conjunction with the endoscope, wherein the therapeutic apparatus includes a suturing device and a control handle, the suturing device is fixed to an apex of the endoscope, and the suturing device includes:
a main housing;
a suture needle, slidingly disposed within the main housing, with one end of the suture needle connected to a suture thread and the other end of the suture needle being a tip;
a needle insertion assembly, disposed within the main housing, the needle insertion assembly including a drive component, a return component, and a needle insertion tooth, the needle insertion tooth being in a separable engagement relationship with the suture needle, wherein
when the drive component acts on the needle insertion tooth and the suture needle that are in an engagement state,
the suture needle is controlled to be inserted, and the return component undergoes deformation when the suture needle is inserted;
when the drive component is released, the engagement state of the needle insertion tooth and the suture needle is released, and the return component drives the needle insertion tooth to return; or
when the drive component acts on the needle insertion tooth that is in a released engagement state,
the needle insertion tooth moves relative to the suture needle, and the return component undergoes deformation when the needle insertion tooth moves relative to the suture needle;
when the drive component is released, the needle insertion tooth engages with the suture needle, and the return component drives the needle insertion tooth and the suture needle to insert; and
an anti-retraction component disposed within the main housing, the anti- retraction component forming a separable engagement relationship with the suture needle to prevent the suture needle from retracting; and
the control handle fixed at an operating end of the endoscope, the drive component of the suturing device is connected to the control handle, the control handle controls the drive component to control the insertion of the suture needle.
